(19) United States
(12) Patent Application Publication
(10) Pub. No.: US 2010/0179186 A1
(43) Pub. Date: Jul. 15, 2010

(54) USE OF A NOVEL ALPHA-7 nAChR ANTAGONIST TO SUPPRESS PATHOGENIC SIGNAL TRANSDUCTION IN CANCER AND AIDS

(75) Inventors: Roger L. PAPKE, Gainesville, FL (US); Peter A. CROOKS, Nicholasville, KY (US); Linda P. DWOSKIN, Lexington, KY (US); Gretchen Lopez HERNANDEZ, Gainesville, FL (US); Zhenfa ZHANG, Lexington, KY (US); Jeffrey S. THINSCHMIDT, Gainesville, FL (US); Guangrong ZHENG, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/578,106

(22) Filed: Oct. 13, 2009

Related U.S. Application Data
(60) Provisional application No. 61/195,820, filed on Oct. 10, 2008.

(51) Int. Cl. A61K 31/47; A61P 35/00; A61K 31/047; A61K 31/03; A61K 31/444

(52) U.S. Cl. 514/308; 514/730; 514/752; 514/751; 514/332; 514/333; 514/314

(57) ABSTRACT

This application provides a method for the use of select quaternary ammonium antagonists to alpha-7 nAChR for the treatment of cancer and HIV and AIDS.

### CROSS-REFERENCE TO RELATED APPLICATIONS

This application claims priority under 35 U.S.C. §119 to U.S. Provisional Application No. 61/195,820, filed on Oct. 10, 2008, the entire content of which is hereby incorporated by reference in its entirety.

### GOVERNMENT RIGHTS

The research and development for inventions described in this application received funding under U19 DA017548, GM57481-01A2, T32-AG000196, and P01 AG10485. The U.S. Government may have rights to various technical features described in this application.

### TECHNICAL FIELD

This application provides a method for the use of select quaternary ammonium antagonists to alpha-7 nAChR for the treatment of cancer and HIV and AIDS.

### BACKGROUND OF THE INVENTION

Cancer is the second leading cause of human death next to coronary disease. Worldwide, millions of people die from cancer every year. As noted by the American Cancer Society, cancer causes the death of well over a half-million people annually in the United States alone, with over 1.2 million new cases diagnosed per year. In the early part of the next century, cancer is predicted to become the leading cause of death. Metastatic disease from a carcinoma is often fatal. Many cancer patients experience physical debilitations following treatment. Furthermore, many cancer patients experience a recurrence.

Like cancer, HIV and AIDS are a very serious health concern worldwide. More than 40 million people are infected worldwide with HIV-1 and an estimated 14,000 new infections occur every day. Over 25 million people have died of HIV/AIDS since the first cases of AIDS were identified in 1981 (CDC, MMWR Morb. Mortal Wkly. Rep., 52:1145-1148, 2003).

It has been discovered that disease states such as cancers and AIDS have a link to neuronal nicotinic acetylcholine receptors (nAChRs). For example, alpha-7 nAChRs has been found in lung cancer cells where their activitation by either natural molecules or compounds in tobacco smoke are shown to promote cancer growth. In addition, it has been found that alpha-7 nAChRs are upregulated in immune cells in AIDS, suggesting that over activation of alpha-7 receptors in macrophages by the AIDS virus protein, may cause premature cell death. The link between cancers, and AIDS, with nAChRs is discussed, for example, in the following references: Egleton et al. (2008) Trends Pharmacol Sci. March; 29(3):151-8; Grozio et al. (2008) Int J Cancer.; 122(8):19115; Zheng et al. (2007) Am J Respir Cell Mol Biol.; 37(6):681-90; Schuller (2007) Prog Exp Tumor Res.; 39:45-63; Carlisle (2007) Pulm Pharmacol Ther.; 20(6):629-41; Chernyaysky et al. (2006) FASEB; 20(12):2093-101; Razani-Boroujerdi et al. (2007) Am J Respir Cell Mol Biol.; 36(1):13-9; Arredondo et al. (2006) J Cancer Res Clin Oncol. 132(10):653-63; Arredondo et al. (2006) Cancer Biol Ther.; 5(5):511-7; Plummer et al. (2005) Respir Res.; 6:29, Ye et al. (2004) J Pharmacol Exp Ther.; 311(1):123-30; Song et al. (2003) Cancer Res.; 63(1):214-21; Jull et al. (2001) J Cancer Res Clin Oncol.; 127(12):707-17; Sciamanna et al. (1997) J Neurochem; 69(6):2302-11; and Santiago Perezi et al., “Upregulation of the a7 nAChR in human macrophages is induced by chronic treatment with hiv1 envelope protein, gp120”, Abstract, 37th meeting of the Society for Neuroscience. San Diego, November 2007.

Thus, antagonists to nAChRs are needed to exploiting the relationship between cancer, AIDS and nAChR activity, and thus provide treatments for these disease states.

### SUMMARY OF THE INVENTION

This application provides a method for the use of selective azaaromatic quaternary ammonium antagonists for the modulation and inhibition of alpha-7 neuronal nicotinic acetylcholine receptors, in order to treat cancer and AIDS.

One embodiment provides a method of treating cancer or AIDS in a subject in need thereof comprising administering a pharmaceutically acceptable amount of a compound of Formula (I)

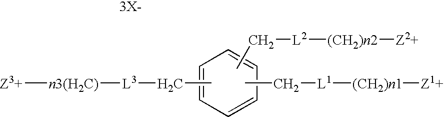

wherein the three side chains attached to the phenyl ring are connected to the 1, 2, and 3 positions; the 1, 2, and 4 positions; or the 1, 3, and 5 positions of the phenyl ring;
wherein:

each X⁻ is independently an organic or inorganic anion;

n1, n2, and n3 are each independently 1, 2, or 3

L¹, L², and L³ are each independently selected from the group consisting of —CH₂CH₂—, cis —CH═CH—, trans —CH═CH—, and —C≡C—;

Z¹, Z², and Z³ are each independently a five or six membered heterocyclic or heteroaryl ring attached through N+ as shown below.

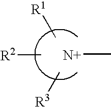

wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

Another embodiment provides a method of treating cancer or AIDS in a subject in need thereof comprising administering a pharmaceutically acceptable amount of a compound of Formula (II)

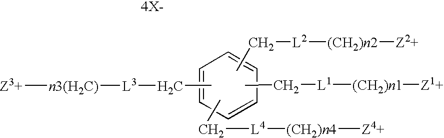

wherein the four side chains attached to the phenyl ring are connected to the 1, 2, 3, and 4 positions; the 1, 3, 4, and 5 positions; or the 1, 2, 4, and 5 positions of the phenyl ring; wherein:

each X⁻ is independently an organic or inorganic anion;

n1, n2, n3 and n4 are each independently 1, 2, or 3

L¹, L², L³ and L⁴ are each independently selected from the group consisting of —CH₂CH₂—, cis —CH═CH—, trans —CH═CH—, and —C≡C—;

Z¹, Z², Z³ and Z⁴ are each independently a five or six membered heterocyclic or heteroaryl ring attached through N+ as shown below.

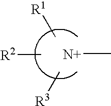

wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

### BRIEF DESCRIPTION OF THE DRAWINGS

FIG. 1 provides azaaromatic quaternary ammonium (AQA) analog structures. 1,3,5,-tri-{5-[1-(2-picolinium)]-pent-1-yn-1-yl}benzene tribromide (tPy2PiB) and 1,3,5-tri-[5-(1-quinolinum)-pent-1-yn-1-yl]-benzene tribromide (tPyQB), and 1,2,4,5-tetra-{5-[1-(3-benzyl)pyridinium]pent-1-yl}benzenetetrabromide (tkP3 BzPB) are shown.

FIG. 2 illustrates the inhibition of rat nAChR responses expressed into Xenopus oocytes. Each panel shows the averaged normalized mean data (±SEM, n≧4) of net charge responses to co-application of ACh and a range of concentrations of tris- and tetrakis-AQA analogs from oocytes expressing rat α4β2, α3β4, or α7 subunits; tPyQB (A), tPy2PiB (B), and tkP3BzPB (C). The data was normalized to responses to ACh alone obtained 5 minutes before the co-application of ACh and antagonist at the indicated concentrations. Open circles correspond to the α4β2 data, while closed circles and squares represent α7 and α4β2, respectively. IC₅₀ values are provided in Table 1.

FIG. 3 shows recovery from tkP3BzPB inhibition in oocytes expressing rat nAChRs. Recovery experiments were performed after 5 minutes wash following application at increasing concentrations of tkP3BzPB for cells expressing α3β4 (A), α4β2 (B) or α7 (C) nAChR. There were no significant effects of tkP3BzPB concentration of the recovery of either α3β4- or α4β2-mediated responses. However there was a tkP3BzPB concentration dependent accumulation of inhibition for the α7-mediated responses. The ICₛₒ estimated for these recovery data was 1.9±0.7 μM, which was not significantly different from the IC₅₀ estimated from the co-application experiments (shown in FIG. 2). D. Determination of recovery rate for tkP3BzPB-induced inhibition of rat α7 nAChR subunits expressed in Xenopus oocytes. Responses to 60 μM ACh co-applied with 1 μM tkP3BzPB were measured at time=0 (arrow). Subsequently, responses to ACh alone were recorded at 5 minute intervals. Data were normalized to original ACh controls. Data represent the mean responses (±SEM, n≧4). Data were fit to an exponential function to estimate the apparent time constant for recovery.

FIG. 4 shows mechanistic studies of AQA analogs-induced inhibition of rat α7 nAChRs expressed into Xenopus oocytes. A. The voltage dependence of α7 nAChR by tPyQB, tPy2PiB, and tkP3BzPB. Cells were held at the indicated holding potentials and then stimulated first by ACh alone and then by ACh plus the tris- and tetrakis-AQA analogs (1 μM for tPyQB, 3 μM for tPy2PiB, and 1 μM for tkP3BzPB). Hyperpolarization did not affect the inhibition produced by tkP3BzPB. However, while 3 μM tPy2PiB produced no significant inhibition at a holding potential of −40 mV, the net charge responses were inhibited 43±3% at a holding potential of −80 mV (***, p<0.001). Likewise, 1 μM tPyQB, which produced 83±2% inhibition when cells were held at −40 mV, produced significantly more inhibition (97.7±0.1%) at −80 mV (***, p<0.001). B. and C. ACh concentration-response curves from cells expressing α7 nAChRs obtained in the presence of either 300 nM tPyQB or 3 μM tPy2PiB (−60 mV), compared to the data for ACh alone. Each point represents data (±SEM) from at least 4 cells, normalized to the maximal response obtainable to ACh alone from the same cell. In order to deliver the compounds effectively in the presence of high concentrations of ACh, which produce very rapid desensitization, the tris-AQA analog was first pre-applied to the bath for 30 seconds and then co-applied with ACh at the indicated concentrations. D. Inhibition and recovery of ACh-evoked responses in oocytes expressing rat α7 nAChRs by tkP3BzPB. Two experimental settings were used; solid bars correspond to the experiments in which a 30 seconds 1 μM tkP3BzPB application preceded ACh and tkP3BzPB co-application and dashed bars correspond to only co-application of ACh and tkP3BzPB. ACh was used at two concentrations (60 and 1000 μM). Data are presented as normalized net charge response. E. To determine if inhibition of α7 by tkP3BzPB was use-dependent, 20 second applications of 3 μM tkP3BzPB were made, either with or without co-application 60 μM ACh. Inhibition of 60 μM ACh-evoked responses was then measured after a 5 minute washout and there was no significant difference in the residual inhibition observed between cells treated with tkP3BzPB alone or tkP3BzPB co-applied with ACh.

FIG. 5 shows inhibition by tkP3BzPB increases with prolonged application to Xenopus oocytes expressing rat α7 nAChRs. A. Representative recordings from a cell tested with the co-application of 100 nM tkP3BzPB and 60 μM ACh. The raw data traces show representative responses of a cell stimulated strongly with 300 μM ACh and then switched to a bath containing 100 nM tkP3BzPB for five minutes. Averaged data from 5 cells (±SEM) are shown in the bar graph below the traces. The net charge data for each cell were normalized relative to the 300 μM ACh responses obtained before the switch to the tkP3BzPB-containing bath solution.

FIG. 6 shows the inhibition of ACh-evoked responses of hippocampal interneurons. Effects of AQAs bath application on α7-mediated responses on hippocampal interneurons are presented in terms of both peak amplitude (A) and net charge (B). Stable baseline responses to the pressure application of 1 mM ACh were obtained from hippocampal interneurons. Cells were stimulated at 30 second intervals, and after four stable responses (1.5 minutes), either 1 μM tPyQB (open circles), tPy2PiB (solid squares), or tkP3BzPB (solid circles) was bath-applied. Solid bar represents the time course of the AQA analog application. C. Representative traces of 1 mM ACh-evoked responses and the inhibition of those responses by 1 μM of tPyQB, tPy2PiB, and tkP3BzPB. Black traces correspond to ACh baseline responses and gray traces correspond to the ACh-evoked responses at the end of AQA application. While tPyQB and tkP3BzPB effectively reduced ACh-evoked responses, tPy2PiB produced no significant reduction of ACh-evoked responses. Horizontal bars represent 250 ms. Vertical bars represent 50 pA. Data represent the averages of 6-7 interneurons.

FIG. 7 shows the differential inhibition of septal neurons by tkP3BzPB co-application. To investigate whether the α7-selectivity of tkP3BzPB would discriminate between the nicotinic components of ACh-evoked responses in septal neurons, a double-barreled picospritzer pressure application system was used with one barrel containing 1 mM ACh and the other containing 1 mM ACh+300 μM tkP3BzPB. A. Representative traces for the tkP3BzPB co-application experiments in septum. Three initial responses to ACh alone were obtained at 30 second intervals and the average of response is shown in the left side of Panel A for both types of cells. Three applications separated by 30 seconds were then made from the barrel containing 1 mM ACh+300 μM tkP3BzPB, and the average of those traces are presented in the middle section of Panel A. After ACh/tkP3BzPB applications, ACh alone was repeatedly applied at 30 second intervals, and the averages of those responses are presented in the right side of Panel A. Horizontal bars represent 250 ms and vertical bars represent 10 pA. B. and C. Bar graph representations of the averaged normalized peak and net charge response, respectively, during co-application (solid bars) and after washout (open bars) for Type I (black bars) and Type II (gray bars) cells. Data represent the average of 11 neurons for Type I and 17 neurons for Type II.

FIG. 8 shows differential inhibition of ACh-evoked responses in septal neurons by tkP3BzPB bath application. Effects of tkP3BzPB bath application on ACh-evoked responses on septum neurons are presented in terms of both peak amplitude (A) and net charge (B). Cells were stimulated at 30 s intervals with 1 mM ACh, and after four stable responses (1.5 minutes) tkP3BzPB was bath applied. Solid bar represents the time course of the 1 μM tkP3BzPB application. Solid circles correspond to the Type I data, while solid squares represent Type II cells. Type II cells were inhibited by tkP3BzPB to a lesser extent. As seen in the inset Type II cells displayed a nicotinic component sensitive to DHβE block (n=3). In the inset open triangles correspond to the average normalized peak amplitude and solid triangles to the average normalized net charge responses. Solid bars represent the time course of the 1 μM tkP3BzPB application, while open bars represent the time course of 1 μM DHβE application. C. Representative traces of 1 mM ACh-evoked responses for Type I and Type II cells in septum and the inhibition of those responses by tkP3BzPB. Black traces correspond to the average of the ACh baseline responses and gray traces correspond to the average of the last four ACh-evoked responses at the end of 1 μM tkP3BzPB application. Horizontal bars represent 250 ms. Vertical bars represent 10 pA for Type I cells and 20 pA for Type II. Data represent the average of 5 neurons for Type I and II neurons for Type II.

### DETAILED DESCRIPTION

In accordance with this detailed description, the following abbreviations and definitions apply. It must be noted that as used herein, the singular forms “a”, “and”, and “the” include plural referents unless the context clearly dictates otherwise. Thus, for example, reference to “a compound” includes a plurality of such compounds and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

The terms “treating”, “treatment”, and the like are used herein to generally mean obtaining a desired pharmacological and physiological effect. More specifically, the reagents described herein which are used to treat a subject with cancer generally are provided in a therapeutically effective amount to achieve any one or more of the following: inhibited tumor growth, reduction in tumor mass, loss of metastatic lesions, inhibited development of new metastatic lesions after treatment has started, or reduction in tumor such that there is no detectable disease (as assessed by, e.g., radiologic imaging, biological fluid analysis, cytogenetics, fluorescence in situ hybridization, immunocytochemistry, colony assays, multiparameter flow cytometry, or polymerase chain reaction). The term “treatment”, as used herein, covers any treatment of a disease in any mammal, particularly a human.

The term “subject” or “patient” as used herein is meant to include a mammal. Preferably the mammal is human.

The term “pharmaceutically effective” as used herein refers to the effectiveness of a particular treatment regime. Pharmaceutical efficacy can be measured based on such characteristics (but not limited to these) as inhibition of tumor growth, reduction of tumor mass, reduction of metastatic lesions as assessed, for example, by radiologic imaging, slowed tumor growth, lack of detectable tumor associated antigens, and the like. Pharmaceutical efficacy can also be measured based on such characteristics (but not limited to these) as inhibition of the HIV virus and/or reduction and eradication of AIDS related symptoms. Additional methods of assessing tumor progression or HIV/AIDS would be known to the treating and diagnosing physicians.

By “pharmaceutically effective amount” is meant an amount of an agent, reagent, compound, composition, or combination of reagents disclosed herein that when administered to a mammal is sufficient to be effective against the cancer or HIV/AIDS.

By the term “tumor” is meant to include both benign and malignant growths or cancer. By the term “cancer”, is meant, unless otherwise stated, both benign and malignant growths. Preferably, the tumor is malignant. The tumor can be a solid tissue tumor such as a melanoma, or a soft tissue tumor such as a lymphoma, a leukemia, or a bone cancer. By the term “primary tumor” is meant the original neoplasm and not a metastatic lesion located in another tissue or organ in the patient's body. By the terms “metastatic disease”, “metastases”, and “metastatic lesion” are meant a group of cells which have migrated to a site distant relative to the primary tumor.

By “AIDS” is meant HIV infection: AIDS, ARC (AIDS related complex), both symptomatic and asymptomatic, and actual or potential exposure to HIV. Accordingly, the treatment of AIDS refers to the inhibition of HIV virus, the prophylaxis or treatment of infection by HIV and the prophylaxis, treatment or the delay in the onset of consequent pathological conditions such as AIDS. The prophylaxis of AIDS, treating AIDS, delaying the onset of AIDS, the prophylaxis of infection by HIV, or treating infection by HIV is defined as including, but not limited to, treatment of a wide range of states of HIV infection: AIDS, ARC (AIDS related complex), both symptomatic and asymptomatic, and actual or potential exposure to HIV.

The term “nicotinic acetylcholine receptor” refers to the endogenous acetylcholine receptor having binding sites for acetylcholine which also bind to nicotine. The term “nicotinic acetylcholine receptor” includes the term “neuronal nicotinic acetylcholine receptor.”

The terms “subtype of nicotinic acetylcholine receptor,” and “nicotinic acetylcholine receptor subtype” refer to various subunit combinations of the nicotinic acetylcholine receptor, and may refer to a particular homomeric or heteromeric complex, or multiple homomeric or heteromeric complexes.

The term “agonist” refers to a substance which interacts with a receptor and increases or prolongs a physiological response (i.e. activates the receptor).

The term “partial agonist” refers to a substance which interacts with and activates a receptor to a lesser degree than an agonist.

The term “antagonist” refers to a substance which interacts with and decreases the extent or duration of a physiological response of that receptor.

The terms “disorder,” “disease,” and “condition” are used inclusively and refer to any status deviating from normal.

The term “central nervous system associated disorders” includes any cognitive, neurological, and mental disorders causing aberrant or pathological neural signal transmission, such as disorders associated with the alteration of normal neurotransmitter release in the brain.

The term “alkyl” refers to straight or branched chain alkyl radicals having 1 to 8 carbon atoms.

The term “cycloalkyl” refers to cyclic ring-containing moieties containing 3 to 8 carbon atoms.

The term “alkylcycloalkyl” refers to alkyl-substituted cycloalkyl groups.

The term “cycloalkylalkyl” refers to cycloalkyl-substituted alkyl groups.

The term “alkenyl” refers to straight or branched chain hydrocarbyl groups having at least one carbon-carbon double bond and having 2 to 10 carbon atoms.

The term “alkynyl” refers to straight or branched chain hydrocarbyl moieties having at least one carbon-carbon triple bond and having 2 to 10 carbon atoms.

The term “aryl” refers to aromatic groups having 6 to 24 carbon atoms.

The term “alkylaryl” refers to alkyl-substituted aryl groups.

The term “arylalkyl” refers to aryl-substituted alkyl groups.

The term “arylalkenyl” refers to aryl-substituted alkenyl groups, and “substituted arylalkenyl” refers to arylalkenyl groups further bearing one or more substituents as set forth above.

The term “arylalkynyl” refers to aryl-substituted alkynyl groups, and “substituted arylalkynyl” refers to arylalkynyl groups further bearing one or more substituents as set forth above.

The term “heterocyclic” refers to cyclic moieties containing one or more heteroatoms as part of the ring structure and having 3 to 24 carbon atoms.

The term “alkylheterocyclic” refers to alkyl-substituted heterocyclic groups.

The term “heterocyclicalkyl” refers to heterocyclic-substituted alkyl groups.

The term “heteroaryl” refers to aromatic groups having 5 to 24 ring atoms wherein the ring contains one or more heteroatoms as part of the ring struture.

The term “alkylheteroaryl” refers to alkyl-substituted heteroaryl groups.

The term “heteroarylalkyl” refers to heteroaryl-substituted alkyl groups.

The term “halogen” refers to fluoride, chloride, bromide or iodide groups.

The present invention provides for the treatment of cancer and HIV/AIDS using quaternary ammonium salts and their use in modulating nicotinic acetylcholine receptors. The particular compounds of the present invention are effective antagonists of alpha-7 nicotinic acetylcholine receptors (nAChRs). Accordingly, the present invention provides the use of select quaternary ammonium salts for the treatment of cancers and AIDS, as well as conditions related thereto.

S(−)-Nicotine (NIC) activates presynaptic and postsynaptic neuronal nicotinic receptors that evoke the release of neurotransmitters from presynaptic terminals and that modulate the depolarization state of the postsynaptic neuronal membrane, respectively. Thus, nicotine produces its effect by binding to a family of ligand-gated ion channels, stimulated by acetylcholine (ACh) or nicotine which causes the ion channel to open and cations to flux with a resulting rapid (millisecond) depolarization of the target cell. Neuronal nicotinic acetylcholine receptors (nAChRs) are distributed throughout the central and peripheral nervous systems (Role et al. (1996), Neuron 16(6):1077-1085; Wonnacott S (1997) TINS 20(2):92-98). Presently, nine neuronal a subunits (α2-α10) and three neuronal β subunits (β2-β4) have been identified and cloned in vertebrate systems. One type of neuronal nAChR is formed by the assembly of α and β subunits, with functional properties depending on both α and β subunits within the receptor complex (Buisson and Bertrand (2002) Trends Pharmacol Sci 23(3):130-136). Additionally, α7, α8, or α9 nAChR subunits can form functional α-bungarotoxin-sensitive homopentamers (Couturier et al. (1990) Neuron 5:847-856; Elgoyhen (1994) Cell 79:705-715; Peng et al., (1994) Mol Pharm 45:546-554; Seguela et al. (1993) J Neurosci 13(2):596-604)). Neuronal nAChRs are involved mostly, but not exclusively, in presynaptic transmission in the central nervous system (CNS). Presynaptic nAChRs have been detected on several cell populations in the brain (e.g. cortex, hippocampus, and cerebellum) where they can modify the excitability of neurons, facilitate neurotransmitter release (ACh, dopamine, noradrenaline, gamma aminobutyric acid (GABA), 5-hydroxytrytamine, and glutamate), among other functions (Dani et al. (2007) Annu Rev Pharmacol Toxicol 47:699-729; Gotti et al. (1997) Progress in Neurobio 53:199-237; Hogg et al. (2003) Rev Physiol Biochem Pharmacol 147:1-46; Wonnacott (1997) TINS 20(2):92-98). For example, presynaptic α7 receptors have been implicated in the regulation of glutamate release in the hippocampus (Gray et al. (1996) Nature 383:713-716).

The therapeutic targeting of neuronal nAChRs is made challenging by the great diversity in their composition, distribution, and pharmacological properties. For instance, in rat medial septum multiple functional nAChR subtypes are expressed that are associated with variations in the neuronal, physiological, and neurotransmitter phenotype. Medial septal neurons that have fast firing rates are likely to be GABAergic, and often have both fast and slow components to their ACh-evoked responses, with the fast component being sensitive to methyllycaconitine (MLA) blockade (Thinschmidt et al. (2005) Neurosci Lett 389(3):163-168). The pharmacological isolation of these nicotinic components is possible with the use of subtype-selective agonists and antagonists. For example, it was previously shown that the amphipathic blocker 2,2,6,6-tetramethylpiperidin-4-yl heptanoate (TMPH) produced a potent and long-lasting inhibition of non-α7 receptors, particularly α4β2 nAChRs, but only transient inhibition of α7 receptors expressed in Xenopus oocytes. Extending those findings, TMPH, which produced long-lasting inhibition of only non-α7 nAChRs, was shown to be useful in the characterization of complex nicotinic response, such as those arising from multiple nAChR subtypes both in the oocyte expression system and in medial septal slices from rat (Papke et al. (2005) Mol Pharmacol 67(6):1977-1990).

Despite the extensive diversity in neuronal nicotinic receptor messenger RNA expression, only a limited number of tools are available to study the pharmacology of native receptors. Radioligands are used in many studies. [³H]NIC appears to label the same sites in the brain as [³H]ACh. It has been estimated that over 90% of [³H]NIC binding in the brain is due to association with the heteromeric receptor that is composed of α4 and β2 subunits. Also abundant in the central nervous system are the homomeric receptors labeled by [³H]methyllycaconitine (MLA), which has high affinity for the α7 nicotinic receptor subtype. Nicotinic receptor subtypes can be studied using functional assays, such as NIC-evoked neurotransmitter release (e.g., [³H]dopamine (DA) release, [³H]norepinephrine (NE) release, [³H]serotonin (5-HT) release, [³H]gamma-aminobutyric acid (GABA) release and [³H]glutamate release) from superfused rat brain slices. Nicotinic receptors are located in the cell body and terminal neurotransmitter release from nerve terminals.

The present nAChR subtype-selective antagonists come from a family of tris- and tetrakis-azaaromatic quaternary ammonium (AQA) compounds. tris-azaaromatic quaternary ammonium (AQA) compounds contemplated by the present invention may be selected from found in U.S areas of these neurotransmitter systems. NIC facilitates application Ser. No. 12/158,192, the contents of which is hereby incorporated by reference in their entirety. tetrakis-azaaromatic quaternary ammonium (AQA) compounds contemplated by the present invention may be selected from those found in U.S. application Ser. No. 12/260,502, the contents of which is hereby incorporated by reference in their entirety.

In one embodiment, a method of treating cancer or AIDS in a subject in need thereof is provided, comprising administering a pharmaceutically acceptable amount of a tris-azaaromatic quaternary ammonium compound. The compound can be a compound of Formula (I)

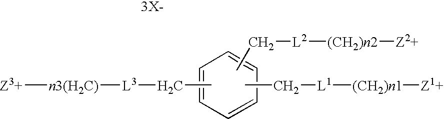

wherein the three side chains attached to the phenyl ring are connected to the 1, 2, and 3 positions; the 1, 2, and 4 positions; or the 1, 3, and 5 positions of the phenyl ring;
wherein:

each X⁻ is independently an organic or inorganic anion;

n1, n2, and n3 are each independently 1, 2, or 3

L¹, L², and L³ are each independently selected from the group consisting of —CH₂CH₂—, cis —CH═CH—, trans —CH═CH—, and —C≡C—;

Z¹, Z², and Z³ are each independently a five or six membered heterocyclic or heteroaryl ring attached through N+ as shown below

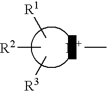

wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring. n1, n2, and n3 can be 2. L¹, L², and L³ can each independently be —CH₂CH₂— or —C≡C—. In one embodiment, L¹, L², and L³ are the same. Z¹, Z², and Z³ can each independently be pyridinyl rings attached through N+ as shown below.

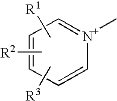

wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring. In one embodiment, two of R¹, R², and R³ are hydrogen and one is alkyl, aryl, alkylaryl, arylalkyl, heterocyclic, alkylheterocyclic, heterocyclicalkyl, alkylheteroaryl, or heteroarylalkyl. In another embodiment, one of R¹, R², and R³ is hydrogen and two of R¹, R², and R³ together with the atoms to which they are attached form a six membered aryl ring.

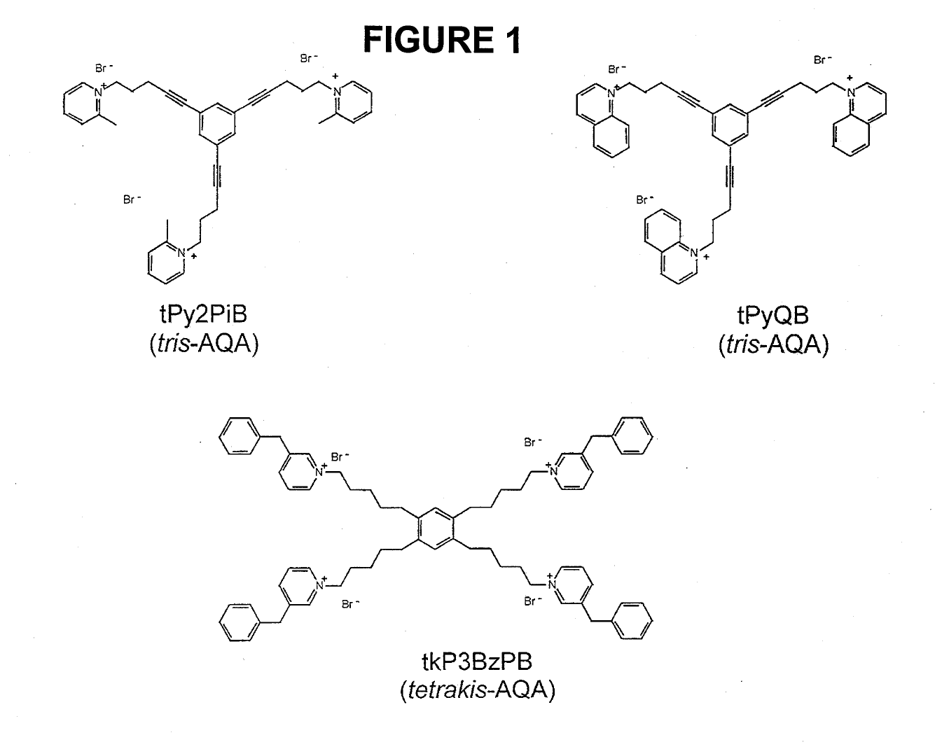

Z¹, Z², and Z³ can be the same. R¹, R², and R³ can each independently be selected from hydrogen, alkyl, aryl, alkylaryl, arylalkyl, or two of R¹, R², and R³ together with the carbon atoms to which they are attached form a six membered aryl ring. In one embodiment, the compound of formula (I) is 1,3,5,-tri-{5-[1-(2-picolinium)]-pent-1-yn-1-yl}benzene tribromide (tPy2PiB). In another embodiment, the compound of formula (I) is 1,3,5-tri-[5-(1-quinolinum)-pent-1-yn-1-yl]-benzene tribromide (tPyQB) (see FIG. 1).

In another embodiment, a method of treating cancer or AIDS in a subject in need thereof is provided comprising administering a pharmaceutically acceptable amount of a tetrakis-azaaromatic quaternary ammonium compound. The compound can be a compound of Formula (II):

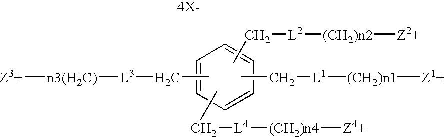

wherein the four side chains attached to the phenyl ring are connected to the 1, 2, 3, and 4 positions; the 1, 3, 4, and 5 positions; or the 1, 2, 4, and 5 positions of the phenyl ring;
wherein:

each X⁻ is independently an organic or inorganic anion;

n1, n2, n3 and n4 are each independently 1, 2, or 3

L¹, L², L³ and L⁴ are each independently selected from the group consisting of —CH₂CH₂—, cis —CH═CH—, trans —CH═CH—, and —C≡C—;

Z¹, Z², Z³ and Z⁴ are each independently a five or six membered heterocyclic or heteroaryl ring attached through N+ as shown below.

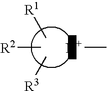

wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring. n1, n2, n3 and n4 can be 2. In one embodiment, L¹, L², L³ and L⁴ can each independently be —CH₂CH₂— or —C≡C—. L¹, L², L³ and L⁴ can be the same.

Z¹, Z², Z³ and Z⁴ can each independently be pyridinyl rings attached through N+ as shown below.

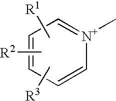

wherein R¹, R², and R³ are each independently selected from hydrogen, alkyl, cycloalkyl, alkenyl, alkynyl, aryl, alkylaryl, arylalkyl, arylalkenyl, arylalkynyl, heterocyclic, alkylheteroaryl, heteroarylalkyl, halo or two of R¹, R², and R³ together with the atoms to which they are attached form a three to six membered cycloalkyl, aryl, or heterocyclic with one to two hetero atoms in the ring. Z¹, Z², Z³ and Z⁴ can be the same.

In one embodiment, two of R¹, R², and R³ are hydrogen and one is alkyl, aryl, alkylaryl, arylalkyl, heterocyclic, alkylheterocyclic, heterocyclicalkyl, alkylheteroaryl, or heteroarylalkyl. In another embodiment, one of R¹, R², and R³ is hydrogen and two of R¹, R², and R³ together with the atoms to which they are attached form a six membered aryl.

R¹, R², and R³ can each independently be selected from hydrogen, alkyl, aryl, alkylaryl, arylalkyl, or two of R¹, R², and R³ together with the carbon atoms to which they are attached form a six membered aryl.

In one embodiment, the compound of formula (II) is 1,2,4,5-tetra-{5-[1-(3-benzyl)pyridinium]pent-1-yl}benzenetetrabromide. (tkP3BzPB) (see FIG. 1).

The tris- and tetrakis-AQA analogs of the present invention show a high selectivity for α7 4 nAChRs. These compounds can block α7-mediated responses and preserve the responsiveness of non-α7 receptors. A preliminary study of a large family of tris-AQA compounds reported that the effectiveness of such compounds at inhibiting α7 nAChR was correlated to the hydrophobicity of the head group (Papke et al. (2006) in 68th Annual Meeting College on Problems of Drug Dependence, Scottsdale, Ariz.). (See also, Lopez-Hernandez et al. (2009) Molec. Pharmacol. 76(3):652-66). Accordingly, one aspect of the invention provides for methods using and compositions comprising azaaromatic quaternary ammonium analogs of the present invention. An azaaromatic quaternary ammonium analogs can be the tris-azaaromatic quaternary ammonium analog compounds, including 1,3,5,-tri-{5-[1-(2-picolinium)]-pent-1-yn-1-yl}benzene tribromide (tPy2PiB) and 1,3,5-tri-[5-(1-quinolinum)-pent-1-yn-1-yl]-benzene tribromide (tPyQB). In another embodiment, the azaaromatic quaternary ammonium analogs may be a tetrakis-azaaromatic quaternary ammonium analog compound, including 2,4,5-tetra-{5-[1-(3 benzyl)pyridinium]pent-1-yl}benzenetetrabromide (tkP3BzPB).

The relative efficacy of tPyQB and tPy2PiB were consistent with that hypothesis. The inhibitory action of the AQA analogs in simple co-application experiments with oocytes expressing α7 nAChRs rank as follows: tPyQB>tkP3BzPB>>tPy2PiB. However, inhibition by tkP3BzPB was not readily reversible. This suggests that the inhibition of α7 nAChR by this compound is qualitatively different from that produced by the tris-AQA analogs, in that it accumulates over time to produce more inhibition than can be measured with a simple co-application protocol. Another indication that the mechanism of inhibition by tkP3BzPB differs from that of the tris-AQA analogs is that inhibition by the tetrakis-AQA compound tkP3BzPB was not voltage-dependent, while inhibition by tPy2PiB and tPyQB was. Lack of voltage-dependence is consistent with the lack of use-dependence observed, and suggests that tkP3BzPB may exert its inhibitory effects by binding to sites in the vestibule of the receptor.

The tris-AQA analogs, tPyQB and tPy2PiB, produced inhibition of α7 ACh-evoked responses that was at least in part non-competitive, consistent with the voltage dependence data which suggests at least some interaction at sites within the membrane's electric field. Although the inhibition of α7 by tPy2PiB and tPyQB was not fully surmountable by increasing ACh concentration, the compounds produced apparent shifts in ACh potency, suggesting that the mechanism of inhibition by these compounds could arise from multiple mechanisms. For example, the structural properties of tPy2PiB (polar head group) as well as its voltage-dependence might suggest that the induced inhibition could arise from direct channel blocking. However, since the effects of tPy2PiB (and tPyQB) were readily reversible and inhibition could only be observed during an ACh application, we could not determine if their inhibitory effects required channel activation (consistent with open channel block), or merely could be measured only during channel activation. On the other hand, the lack of both voltage- and use-dependence for tkP3BzPB suggests that its antagonist properties arise from binding to other sites that are different from either the ACh binding sites or the ion channel.

The azaaromatic quaternary ammonium analogs of the present invention, as modulators and inhibitors of nAChRs, are indicated as useful in the treatment and/or prophylaxis of various diseases and conditions, including cancer and AIDS. Accordingly, the present invention provides a method of treating a subject suffering from cancer or AIDS comprising administering to the subject an effective amount of the azaaromatic quaternary ammonium analogs of the present invention.

In one aspect of the invention, the methods and compositions disclosed herein can be used to inhibit or slow the progression of malignancies. These malignancies can be solid or soft tissue tumors. Soft tissue tumors include bone cancers, lymphomas, and leukemias. Another aspect of the invention is to use the methods and compositions to inhibit or prevent metastases or metastatic progression. The azaaromatic quaternary ammonium analogs can be used alone, in combination with each other, or in combination with other cancer modalities, such as but not limited to chemotherapy, surgery, radiotherapy, hyperthermia, immunotherapy, hormone therapy, biologic therapy (e.g., immune effector mechanisms resulting in cell destruction, cytokines, immunotherapy, interferons, interleukin-2, cancer vaccine therapy, and adoptive therapy), and drugs to ameliorate the adverse side effects of such cancer modalities.

The term cancer embraces a collection of malignancies with each cancer of each organ consisting of numerous subsets. Typically, at the time of cancer diagnosis, “the cancer” consists in fact of multiple subpopulations of cells with diverse genetic, biochemical, immunologic, and biologic characteristics. Cancers to be treated can include, but are not limited to, melanomas (e.g., cutaneous melanoma, metastatic melanomas, and intraocular melanomas), prostate cancer, lymphomas (e.g., cutaneous T-cell lymphoma, mycosis fungicides, Hodgkin's and non-Hodgkin's lymphomas, and primary central nervous system lymphomas), leukemias (e.g., pre-B cell acute lymphoblastic leukemia, chronic and acute lymphocytic leukemia, chronic and acute myelogenous leukemia, adult acute lymphoblastic leukemia, mature B-cell acute lymphoblastic leukemia, prolymphocytic leukemia, hairy cell leukemia, and T-cell chronic lymphocytic leukemia), and metastatic tumors which exhibit these proteins on the cell surface. For example, the cancer may be a lung cancer.

Also contemplated for treatment with the methods, combination therapies, and compositions disclosed herein is the treatment of metastatic cancer. Cancers typically begin their growth in only one location in the tissue of origin. As the cancer progresses, the cancer may migrate to a distal location in the patient. For example, a cancer beginning in the prostate may migrate to the lung. Other locations common for metastatic disease and that are contemplated herein include metastatic cancer to the brain, lung, liver, and bone. For additional details on the mechanism and pathology of tumor metastasis, see Isaiah J. Fidler, “Molecular Biology of Cancer: Invasion and Metastasis,” in CANCER: PRINCIPLES & PRACTICE OF ONCOLOGY 135-152 (Vincent T. DeVita et al., editors, 5th ed., 1997).

The present invention also provides a method of treatment of AIDS. The compounds of the present invention are useful in the inhibition of HIV virus, the prophylaxis or treatment of infection by HIV and the prophylaxis, treatment or the delay in the onset of consequent pathological conditions such as AIDS. The prophylaxis of AIDS, treating AIDS, delaying the onset of AIDS, the prophylaxis of infection by HIV, or treating infection by HIV is defined as including, but not limited to, treatment of a wide range of states of HIV infection: AIDS, ARC (AIDS related complex), both symptomatic and asymptomatic, and actual or potential exposure to HIV.

The compounds of the present invention can be delivered directly or in pharmaceutical compositions along with suitable carriers or excipients, as is well known in the art. For example, a pharmaceutical composition of the invention may include a conventional additive, such as a stabilizer, buffer, salt, preservative, filler, flavor enhancer and the like, as known to those skilled in the art. Exemplary buffers include phosphates, carbonates, citrates and the like. Exemplary preservatives include EDTA, EGTA, BHA, BHT and the like.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences.

Suitable routes of administration may, for example, include oral, rectal, transmucosal, nasal, or intestinal administration and parenteral delivery, including intramuscular, subcutaneous, intramedullary injections, as well as intrathecal, direct intraventricular, intravenous, intraperitoneal, intranasal, or intraocular injections. In addition, the agent or composition thereof may be administered sublingually or via a spray, including a sublingual tablet or a sublingual spray. The agent or composition thereof may be administered in a local rather than a systemic manner. For example, a suitable agent can be delivered via injection or in a targeted drug delivery system, such as a depot or sustained release formulation.

The pharmaceutical compositions of the present invention may be manufactured by any of the methods well-known in the art, such as by conventional mixing, dissolving, granulating, dragee-making, levigating, emulsifying, encapsulating, entrapping, or lyophilizing processes. As noted above, the compositions of the present invention can include one or more physiologically acceptable carriers such as excipients and auxiliaries that facilitate processing of active molecules into preparations for pharmaceutical use.

Proper formulation is dependent upon the route of administration chosen. For injection, for example, the composition may be formulated in aqueous solutions, preferably in physiologically compatible buffers such as Hanks' solution, Ringer's solution, or physiological saline buffer. For transmucosal or nasal administration, penetrants appropriate to the barrier to be permeated are used in the formulation. Such penetrants are generally known in the art. In a preferred embodiment of the present invention, the present compounds are prepared in a formulation intended for oral administration. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Also, wetting agents such as sodium dodecyl sulfate may be included.

Pharmaceutical preparations for oral administration include tablets, caplets, capsules, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

The compounds of the present invention can be administered transdermally, such as through a skin patch, or topically. In one aspect, the transdermal or topical formulations of the present invention can additionally comprise one or multiple penetration enhancers or other effectors, including agents that enhance migration of the delivered compound. Transdermal or topical administration could be preferred, for example, in situations in which location specific delivery is desired. Methods of transdermal delivery include microneedle transdermal delivery. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges of, for example, gelatin, for use in an inhaler or insufflator may be formulated. These typically contain a powder mix of the compound and a suitable powder base such as lactose or starch.

Compositions formulated for parenteral administration by injection, e.g., by bolus injection or continuous infusion can be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents. Formulations for parenteral administration include aqueous solutions or other compositions in water-soluble form.

Suspensions of the active compounds may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use. As mentioned above, the compositions of the present invention may also be formulated as a depot preparation. Such long acting formulations may be administered by implantation (for example, subcutaneously or intramuscularly) or by intramuscular injection. Thus, for example, the present compounds may be formulated with suitable polymeric or hydrophobic materials (for example as an emulsion in an acceptable oil) or ion exchange resins, or as sparingly soluble derivatives, for example, as a sparingly soluble salt.

For any composition used in the present methods of treatment, a therapeutically effective dose can be estimated initially using a variety of techniques well known in the art. For example, in a cell culture assay, a dose can be formulated in animal models to achieve a circulating concentration range that includes the IC₅₀ as determined in cell culture. Dosage ranges appropriate for human subjects can be determined, for example, using data obtained from cell culture assays and other animal studies. For example, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner.

A therapeutically effective dose of an agent refers to that amount of the agent which results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The amount of agent or composition administered will, of course, be dependent on a variety of factors, including the sex, age, and weight of the subject being treated, the severity of the affliction, the manner of administration, and the judgment of the prescribing physician. Compounds of the present invention will generally be administered in an amount ranging from about 1×10⁻⁵ to 100 mg/kg/day, with amounts in the range of about 1×10⁻² to 1 mg/kg/day being preferred.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein, and are specifically contemplated.

In some embodiments of the present invention, one or more compounds of the present invention are administered in combination with one or more other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. For example, compounds of the present invention may be administered in combination with one or more other modulators of nAChRs. Alternatively or additionally, compounds of the present invention, in forms as described herein, may be administered in combination with one or more other anti-cancer or anti-viral agent, or other pharmaceutically active agents.

In some embodiments, the compounds of the present invention are administered together with another pharmaceutically active agent in a single administration or composition. In some embodiments, a composition comprising an effective amount of a compound of the present invention and an effective amount of another pharmaceutically active agent within the same composition can be administered. In another embodiment, a composition comprising an effective amount of the compounds of the present invention and a separate composition comprising an effective amount of another pharmaceutically active agent can be concurrently administered. Thus, in some embodiments, the invention provides a composition comprising an effective amount of the a compound of the present invention and a pharmaceutically acceptable carrier. In some embodiments, the composition further comprises a second pharmaceutically active agent.

In another embodiment, the invention refers to a pharmaceutical composition containing one or more compounds of the present invention, in association with pharmaceutically acceptable carriers and excipients. The pharmaceutical compositions can be in the form of solid, semi-solid or liquid preparations, preferably in form of solutions, suspensions, powders, granules, tablets, capsules, syrups, suppositories, aerosols or controlled delivery systems. The compositions can be administered by a variety of routes, including oral, transdermal, subcutaneous, intravenous, intramuscular, rectal and intranasal, and are preferably formulated in unit dosage form, each dosage containing from about 1 to about 1000 mg of the active ingredient. The compounds of the invention can be in the form of free bases or as acid addition salts. The invention also includes separated isomers and diastereomers of the compounds. The principles and methods for the preparation of pharmaceutical compositions are described for example in Remington's Pharmaceutical Science, Mack Publishing Company, Easton Pa.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound (and/or one or more metabolites thereof) into the bloodstream. The mode of administration may be left to the discretion of the practitioner. In some embodiments, provided pharmaceutical compositions are administered orally; in some embodiments, provided pharmaceutical compositions are administered intravenously. For example, it can be desirable to introduce a compound into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir. Pulmonary administration can also be employed, e.g., by use of an inhaler or nebulizer, and formulation with an aerosolizing agent, or via perfusion in a fluorocarbon or synthetic pulmonary surfactant.

One or more compounds of the present invention can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

As depicted in the examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### EXAMPLES

### Example 1

### Chemicals

tris- and tetrakis-AQA analogs were prepared as previously described (Dwoskin et al. (2008) J Pharmacol Exp Ther 326(2):563-576; Zheng et al. (2007) Bioorg Med Chem Lett 17(24):6701-6706). All other chemicals for electrophysiology were obtained from Sigma Chemical Co. (St. Louis, Mo.).

nAChR Expression in Xenopus oocytes

For recombinant nAChR studies, mature (>9 cm) female Xenopus laevis African frogs (Nasco, Ft. Atkinson, Wis.) were used as a source of oocytes. Prior to surgery, frogs were anesthetized by placing the animal in a 1.5 g/l solution of MS222 (3-aminobenzoic acid ethyl ester) for 30 min. Oocytes were removed from an incision made in the abdomen. All procedures involving frogs were approved by the University of Florida Institutional Animal Care and Use Committee (IACUC).

To remove the follicular cell layer, harvested oocytes were treated with 1.25 mg/ml type 1 collagenase (Worthington Biochemicals, Freehold, N.J.) for 2 h at room temperature in calcium-free Barth's solution with a composition in mM of: 88 NaCl, 1 KCl, 0.33 MgSO₄, 2.4 NaHCO₃, 10 HEPES (pH 7.6), and 50 mg/l gentamicin sulfate. Stage 5 oocytes were then isolated and injected with 50 nl (5-20 ng) each of the appropriate subunit cRNAs. The rat neuronal nAChR clones were obtained from Dr. Jim Boulter (UCLA, Los Angeles, Calif.). After linearization and purification of cloned cDNAs, RNA transcripts were prepared in vitro using the appropriate mMessage mMachine kit from Ambion (Austin, Tex.).

Voltage-clamp Recording in Xenopus oocytes Expressing nAChRs

Experiments were conducted using OpusXpress 6000A (Molecular Devices, Union City Calif.). Each oocyte received initial control applications of ACh, then co-applications of the ACh and the experimental drugs, and then a follow-up control application of ACh. The control ACh concentrations for α7 and α4β2 receptors were 60 μM and 10 μM, respectively, and 100 μM for α3β4 receptors. Both the peak amplitude and net charge of the responses were measured for each drug application (Papke and Papke, 2002) and calculated relative to the preceding ACh control responses to normalize the data, compensating for the varying levels of channel expression among the oocytes. Net charge values were used to report inhibitory effects. Competition experiments were conducted by generating concentration-response curves either to ACh applied alone or in the presence of the AQA analog. Responses were initially normalized to the ACh control response values and then adjusted to reflect the experimental drug responses relative to the ACh maximums. Means and standard errors (SEM) were calculated from the normalized responses of at least four oocytes for each experimental concentration. Concentration-response data were fit to the Hill equation assuming negative Hill slopes.

### Brain Slice Preparation and Patch-clamp Recording

All procedures involving rats were approved by the University of Florida IACUC and were in accord with the NIH Guide for the Care and Use of Laboratory Animals. Male Sprague Dawley rats (post-natal day 12-25) were anesthetized with Halothane (Halocarbon Laboratories, River Edge N.J.) and swiftly decapitated. Transverse (300 μM) whole brain slices were prepared using a vibratome (Pelco, Redding, Calif.) and then placed in a high Mg²⁺/low Ca²⁺ ice-cold artificial cerebral spinal fluid (ACSF) containing (in mM) 124 NaCl, 2.5 KCl, 1.2 NaH₂PO₄, 2.5 MgSO₄, 10 D-glucose, 1 CaCl₂, and 25.9 NaHCO₃, saturated with 95% O₂-5% CO₂. Slices were incubated at 30° C. for 30 minutes and then left at room temperature until they were transferred to a submersion chamber (Warner Instruments, Hamden, Conn.) for recording. During experiments, slices were perfused at a rate of 2 ml/min with normal ACSF containing (in mM) 126 NaCl, 3 KCl, 1.2 NaH₂PO₄, 1.5 MgSO₄, 11 D-glucose, 2.4 CaCl₂, 25.9 NaHCO₃, and 0.004 atropine sulfate, saturated with 95% O₂-5% CO₂ at 30° C. Cells were visualized with infrared differential interference contrast microscopy using a Nikon E600FN microscope. Patch-clamp recording pipettes were pulled from borosilicate glass (Sutter Instruments, Novato, Calif.) using a Flaming/Brown micropipette puller (P-97; Sutter Instruments, Novato, Calif.). Recording pipettes were filled with an internal solution of (in mM) 125 K-gluconate, 1 KCl, 0.1 CaCl₂, 2 MgCl₂, 1 EGTA, 2 MgATP, 0.3 Na₃GTP, and 10 HEPES (pH adjusted to 7.3 with KOH). The resistance of the recording pipette when filled with the internal solution was 3-5 Ma Cells were held at −70 mV, and a −10 in V/10 ms test pulse was used to determine access resistance, input resistance, and whole-cell capacitance. Cells with access resistances >60 MΩ or those requiring holding currents >200 pA were not included in the final analyses. Signals were digitized using an Axon Digidata1322A and sampled at 20 kHz on a Dell computer using Clampex version 8 or 9. Data analysis was done with Clampfit version 8 or 9 (Axon Instruments, Union City, Calif.), Excel 2000 (Microsoft, Seattle, Wash.), and GraphPad/Prism version 4.02 (Graphpad Software, San Diego, Calif.). Data are reported as mean±SEM. Statistical analyses were performed using two-tailed Student's t-test and one-way analysis of variance (ANOVA).

### Drug Application in Brain Slice Preparations

Local somatic applications of ACh (1 mM pipette concentration) were made using single- or double-barrel glass pipettes attached to a picospritzer (General Valve, Fairfield, N.J.) with Teflon tubing (10-20 psi for 5-15 ms). ACh was alternately applied every 30 s. Co-application experiments were performed using a double-barrel pressure application pipette in which one side had 1 mM ACh and the other side had 1 mM ACh+300 μM tkP3BzPB, each one alternately applied with a 30 s interstimulus interval. Single-barrel pipettes were pulled from borosilicate glass with an outer diameter (o.d.) and inner diameter (i.d.) of 1.5 mm and 0.86 mm, respectively (Sutter Instrument, Novato, Calif.). Pipette opening size of the single barrel was typically 2-3 μm. Double-barrel pipettes were pulled from borosilicate theta glass with an o.d. of 1.5 mm; pipette opening size was around 3-4 μm. The application pipette was usually placed within 10-15 μm of the cell soma.

In experiments in which AQA analogs were bath-applied, for each cell, four ACh baseline-evoked responses were recorded before bath application of the antagonist. ACh-evoked responses were then recorded for 13-22 min in the presence of the AQA analog. Each analog was bath-applied at a final concentration of 1 μm. In some septum experiments, dihydro-β-erythroidine (DHβE) was also bath-applied at a final concentration of 1 μm.

When pipettes were loaded with 1 mM ACh, the average net charge of evoked responses did not differ significantly between single- and double-barrel experiments (data not shown). Experiments conducted to describe the error produced by alternating pressure applications using double-barrel pipettes showed an 85±8% (n=5) correspondence in the peak amplitudes between the agonist applications from the two barrels (data not shown). In previous experiments, it was determined that pressure application from a pipette containing 1 mM ACh delivered an effective concentration of approximately 30 μm to the surface of the cell (Lopez-Hernandez et al. (2007) Neuropharmacology 53(1):134-144).

Inhibition of Rat nAChR Responses Expressed into Xenopus oocytes

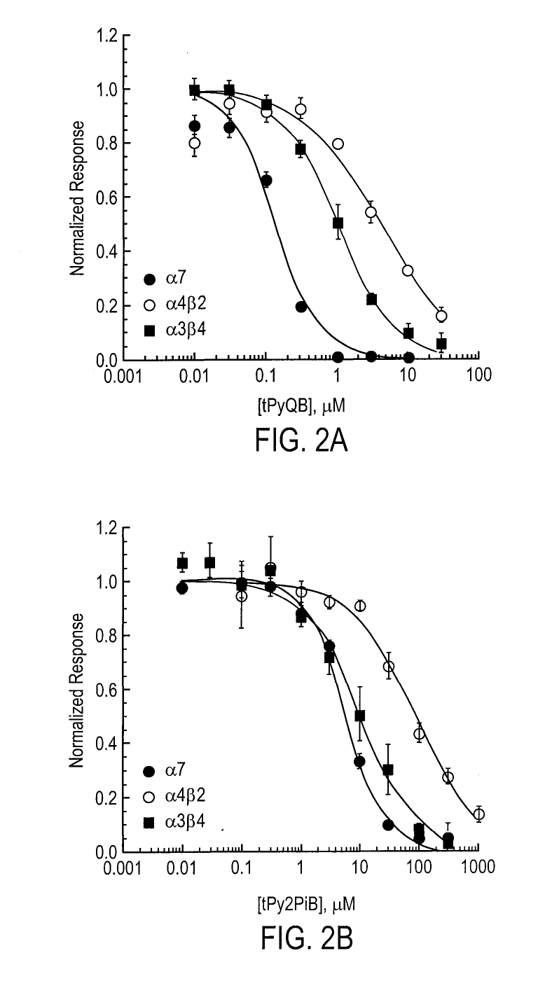

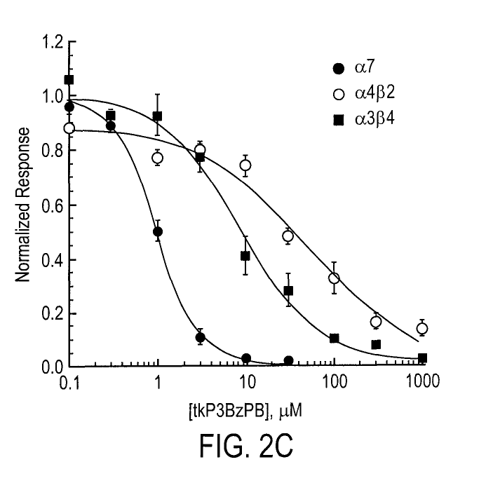

The structures for the quaternary ammonium analogs tPyQB, tPy2PiB, and tkP3BzPB are presented in FIG. 1. These AQA-analogs were tested on representative combinations of rat neuronal nAChR α and β subunits (α4β2 and α3β4) and α7 homomeric receptor, expressed into Xenopus oocytes (FIG. 2 and Table 1). While α3β4 nAChRs represent a minimal model for ganglionic nicotinic receptors, α4β2 and α7 nAChRs are the two predominant subtypes of nicotinic receptors in the CNS. All three AQA analogs were most potent for inhibiting α7 nAChRs compared to the other subunits tested. The IC₅₀ value of tPyQB for oocytes expressing α7 subunits was 0.13±0.02 μM, as determined with a simple co-application protocol, while the IC₅₀ values for tPy2PiB and tkP3BzPB were 6.3±0.6 μM and 1.0±0.1 μM, respectively.

Recovery from Inhibition in Xenopus oocytes Expressing Rat nAChRs

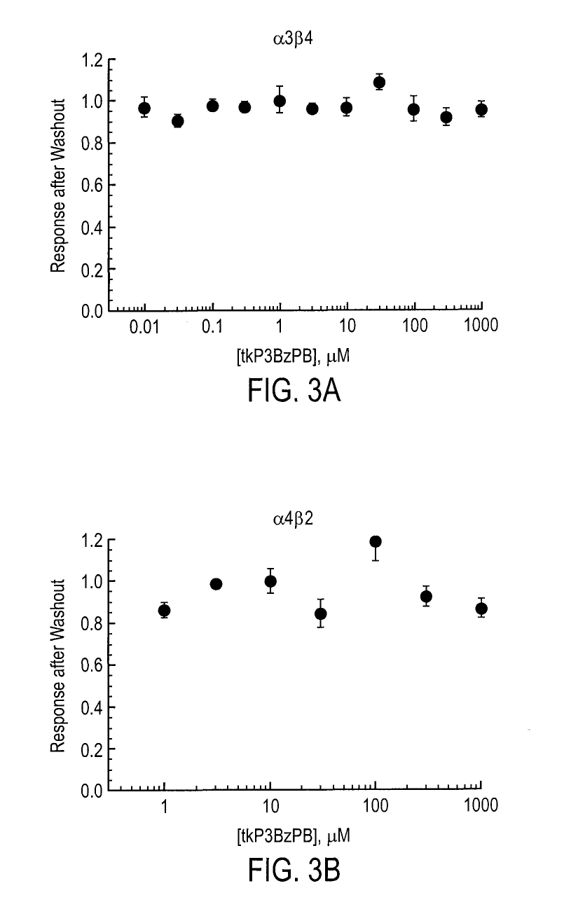

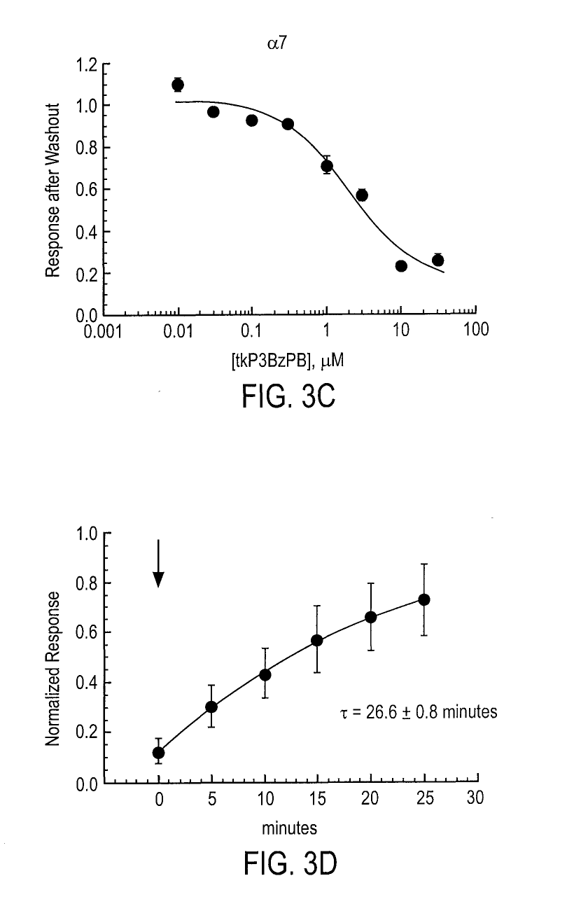

The protocol involved alternating applications of ACh alone and co-applications of antagonists plus ACh to sets of oocytes. In most cases, cells could be tested repeatedly with increasing concentrations of antagonist since responses to ACh alone recovered fully after the co-applications of ACh and antagonist. This was true for all three receptor subtypes tested when using either tPyQB or tPy2PiB. However, a difference in recovery was noted for α7 expressing cells treated with tkP3BzPB. As shown in FIGS. 3 (A and B), α3β4 and α4β2 nAChRs showed no significant residual inhibition 5 min after washout of tkP3BzPB at any of the concentrations tested. However, α7 receptors exhibited decreasing recovery with increasing tkP3BzPB concentrations (FIG. 3C). Therefore, fresh sets of cells were required for each concentration of tkP3BzPB >1 μM, in order to evaluate the inhibition produced by tkP3BzPB and the amount of subsequent recovery. Due to slow recovery after the co-application of ACh and tkP3BzPB, repeated or prolonged applications of tkP3BzPB would be expected to produce greater inhibition than produced by a single co-application.

In order to evaluate the actual rate at which α7 nAChRs recovered from tkP3BzPB inhibition, responses to ACh alone were recorded after 1 μM tkP3BzPB was co-applied with 60 μM ACh. The responses obtained after increasing periods of washout were compared to original ACh controls. The recovery time constant for tkP3BzPB was 26.6±0.8 min (FIG. 3D).

Mechanistic Studies of Inhibition in Xenopus oocytes Expressing Rat nAChR5

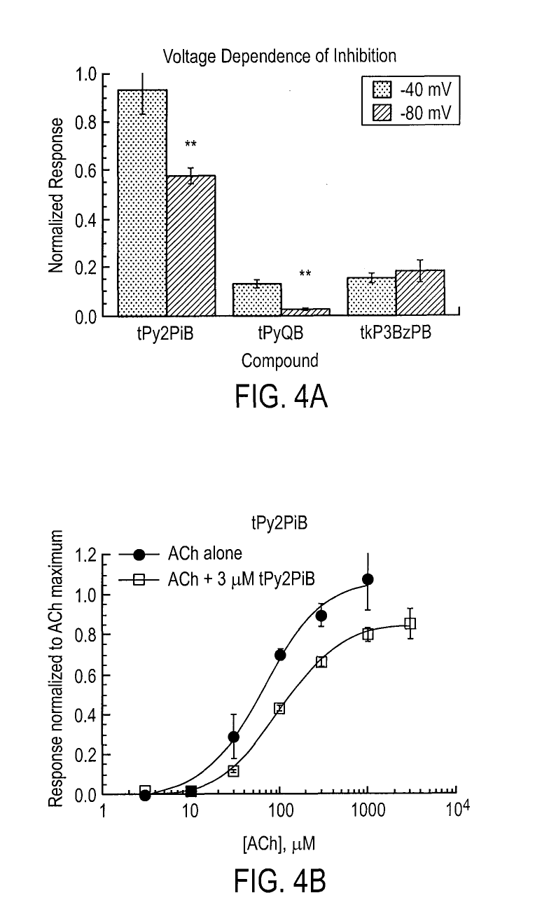

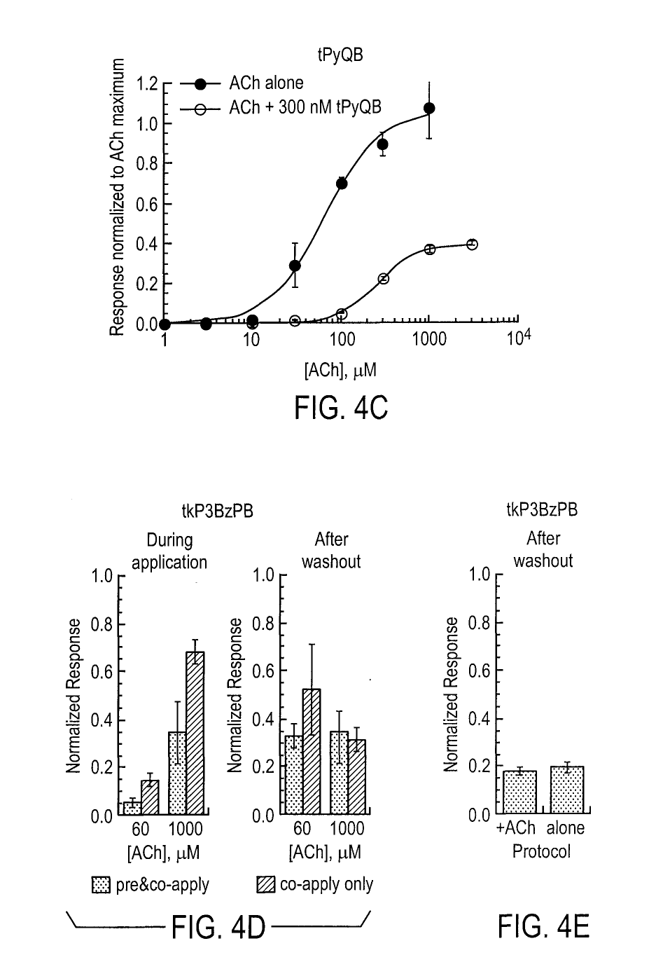

The inhibition of α7 nAChRs by tPyQB, tPy2PiB, or tkP3BzPB was voltage dependent (FIG. 4A) was investigated. Cells were held at either −40 or −80 mV and stimulated first with 60 μM ACh alone or 60 μM ACh plus either 300 nM tPyQB, 3 μM tPy2PiB, or 1 μM tkP3BzPB. There was no significant difference in the inhibition of α7 receptors by tkP3BzPB at these two voltages. However, there was a significant effect of voltage on the inhibition by tPyQB and tPy2PiB.

Additionally, co-application experiments were conducted in Xenopus oocytes expressing rat α7 nAChRs. ACh concentration-response studies of α7 receptors (net charge) were conducted in the presence of either 300 nM of the high potency inhibitor tPyQB or 3 μM of the less potent antagonist tPy2PiB, and compared to the responses to ACh alone (FIGS. 4 B and C). The data obtained with ACh alone were well fit with an Iₘₐₓ=1 (by definition) and an EC₅₀=65±9 μM. In the presence of tPyQB, the Iₘₐₓ was reduced to 0.40±0.01, and the EC₅₀ was 267±8 μM (FIG. 4C). In the presence of 3 μM tPy2PiB the Iₘₐₓ was reduced to 0.84±0.02, with an EC₅₀ of 105±9 μM (FIG. 4B). In the case of tPyQB and tPy2PiB, both compounds produced a depression of the maximal response of agonist dose-response curves and this inhibition was not completely overcome by increasing ACh concentrations. This data is consistent with noncompetitive inhibition. However, tPyQB also produced a larger rightward shift of the dose-response curve, while there was a small shift in EC₅₀ value for ACh in the presence of tPy2PiB. These changes in EC₅₀ values in the presence of tPyQB and tPy2PiB suggest a more complex mechanism than just simple voltage-dependent channel block.

Due to the fact that there was poor recovery of α7 responses after application of tkP3BzPB, it was not practical to generate full ACh concentration response curves in the presence of this compound. Nonetheless, whether inhibition by tkP3BzPB could be surmounted by high concentrations of ACh, consistent with competitive inhibition was determined (FIG. 4D). Because high concentrations of ACh evoke responses that are more rapid than solution exchange (Papke et al. (2000) Eur J Pharmacol 393(1-3):179-195; Papke et al. (2002) Br J of Pharm 137(1):49-61), in order for tkP3BzPB to be even present at full concentration at the time of the peak of 1 mM ACh-evoked current, tkP3BzPB was first pre-applied for 10 s and then co-applied. For comparison, we also conducted simple co-application experiments. As seen in FIG. 4D, there was less inhibition of α7-mediated ACh-evoked responses by tkP3BzPB when the ACh concentration was 1 mM than when it was 60 μM, in both experimental settings (pre- and co-application and with co-application only). However, the concentration-dependent difference in initial inhibition (i.e. the inhibition measured during the initial co-application) was not apparent in the responses measured after washout. This observation was consistent with the hypothesis that the onset of inhibition by tkP3BzPB is relatively slow compared to the kinetics of the α7 response evoked by 1 mM ACh, at least in regard to the persistent inhibition measured after washout, and indicates that inhibition accumulated throughout the tkP3BzPB application regardless of whether ACh was present at high or low concentration, apparently reaching the same equilibrium inhibition prior to the full washout of the drug from the chamber. This suggested that tkP3BzPB might be able to produce inhibition in the absence of channel activation. If this is the case then inhibition will depend on both tkP3BzPB concentration and the amount of time that the antagonist is present. In the co-application experiments tkP3BzPB was present for 20 seconds, the same duration as the agonist pulse. When using 60 μM ACh, the α7 receptors continue to respond throughout the entire 20 second application, as so are exposed to the antagonist for a full 20 seconds (30 seconds when the drug was also preapplied). In contrast, α7 responses evoked by 1 mM rapidly reach a peak, long before the agonist and antagonist co-application is even complete (Papke and Thinschmidt, 1998). Therefore during the co-application of 1 mM and tkP3BzPB inhibition is measured after a very brief exposure to the antagonist, too soon for inhibition to equilibrate to the degree that it did during the longer 60 μM ACh-evoked responses. This effect was diminished with the pre-application protocol.

To test the hypothesis that the inhibition of α7 by tkP3BzPB was use independent (i.e. that inhibition did not require channel activation), 20 second applications of 3 μM tkP3BzPB were made, either with or without co-application 60 μM ACh. Inhibition of 60 μM ACh-evoked responses was then measured after a 5 minute washout. As shown in FIG. 4E, there was no significant difference in the residual inhibition of α7-mediated responses, whether tkP3BzPB was applied alone for 20 s or co-applied with 60 μM ACh. Therefore, the persistent inhibition of α7 nAChRs induced by tkP3BzPB does not require any channel activation; was not use-dependent. Additionally, the time constant of recovery after the application of 3 μM tkP3BzPB alone, measured with repeated applications of 60 μM ACh, was 76±3 minutes.

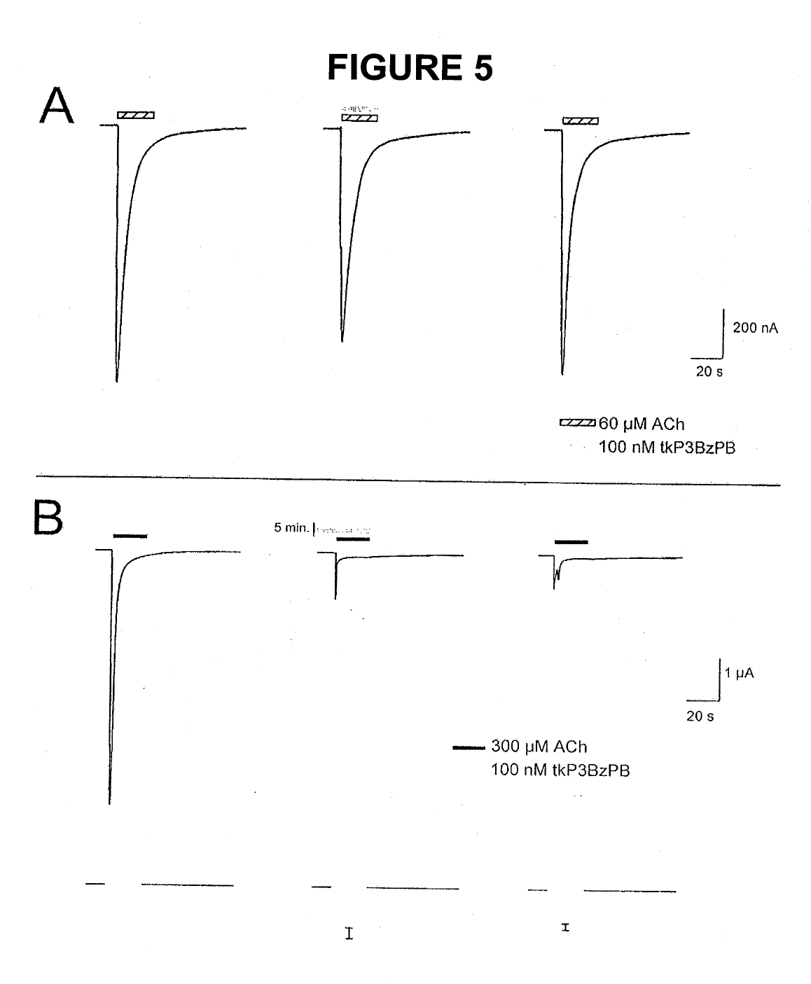

The use-independence, as shown in FIG. 4E, and slow kinetics of recovery shows that tkP3BzPB would show increased potency at α7 nAChRs with prolonged application. With the typical co-application protocol (FIG. 5A), 100 nM tkP3BzPB produced virtually no inhibition of 60 μM ACh-evoked responses either during the brief co-application (FIG. 2, bottom panel), or following the washout period (FIG. 3C). In order to confirm that prolonged application of 100 nM tkP3BzPB could produce substantial inhibition of ACh-evoked responses, α7-expressing cells were stimulated with 300 μM ACh, a concentration which produces a maximal net charge response, and then switched the bath solution to one containing 100 nM tkP3BzPB for 5 minutes (pre-incubation period) before co-applying 100 nM tkP3BzPB and 300 μM ACh. As shown in FIG. 5B, this protocol produced about 80% inhibition of the ACh-evoked responses that persisted through an additional 5 minute washout period.

Activity of tris- and tetrakis-AQA Analogs on Native α7 Receptors on Rat Hippocampal Interneurons

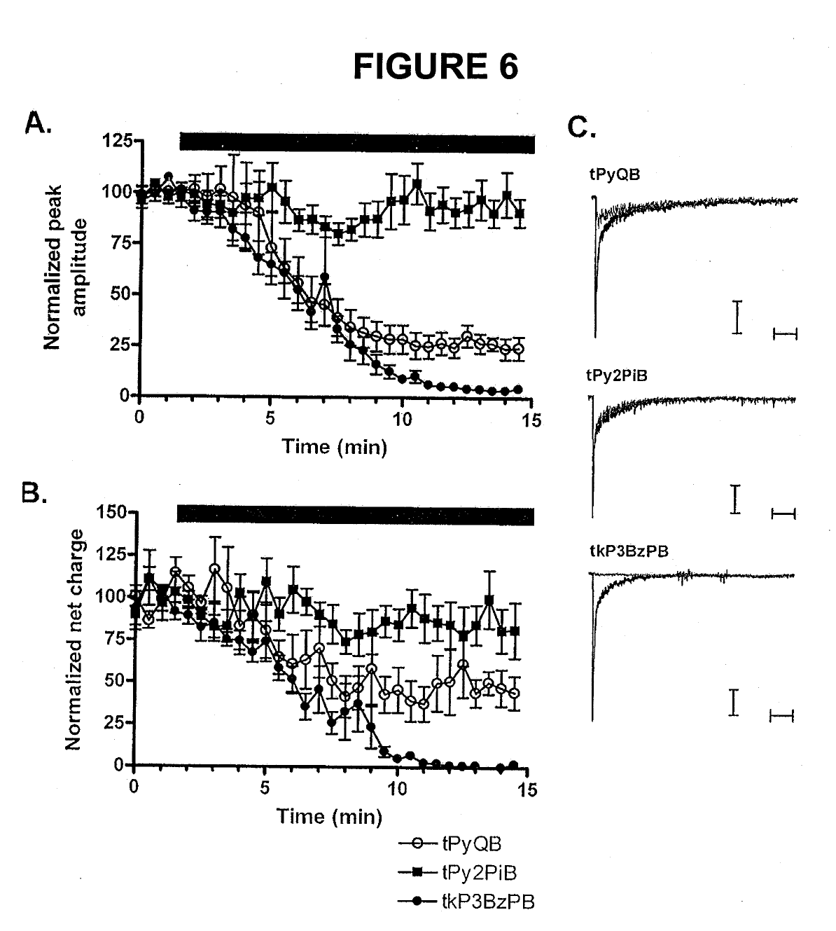

Interneurons in CA1 stratum radiatum of the rat hippocampus show robust responses to the pressure application of ACh which are mediated primarily by α7 type nAChRs (Alkondon et al. (1999) J Neurosci 19(7):2693-2705; Frazier et al. (1998) J Neurosci 18(4):1187-1195; Thinschmidt et al. (2005) Exp Neurol 195(2):342-352). Stable ACh-evoked responses were obtained from hippocampal interneurons in fresh brain slices and then applied 1 μM tPyQB, tPy2PiB, or tkP3BzPB to the bath. In oocytes expressing α7 nAChRs, 1 μM tPyQB was shown to completely inhibit 60 μM ACh-evoked responses (FIG. 2A), thus, this concentration was selected for comparing the inhibition of ACh-evoked responses induced by the AQA analogs in hippocampal interneurons. Consistent with the oocyte data, the ACh-evoked responses of hippocampal interneurons were effectively reduced by tPyQB and tkP3BzPB, but not by tPy2PiB (FIG. 6). Only 4±0.2% of the baseline peak response and 0.4±0.7% of the baseline net charge response remained after tkP3BzPB bath application. Thus tkP3BzPB produced a larger inhibition of ACh-evoked responses in rat hippocampal interneurons in terms of both peak amplitude and net charge responses compared to the other AQA analogs.

Differential Inhibition of Medial Septal Neurons by tkP3BzPB

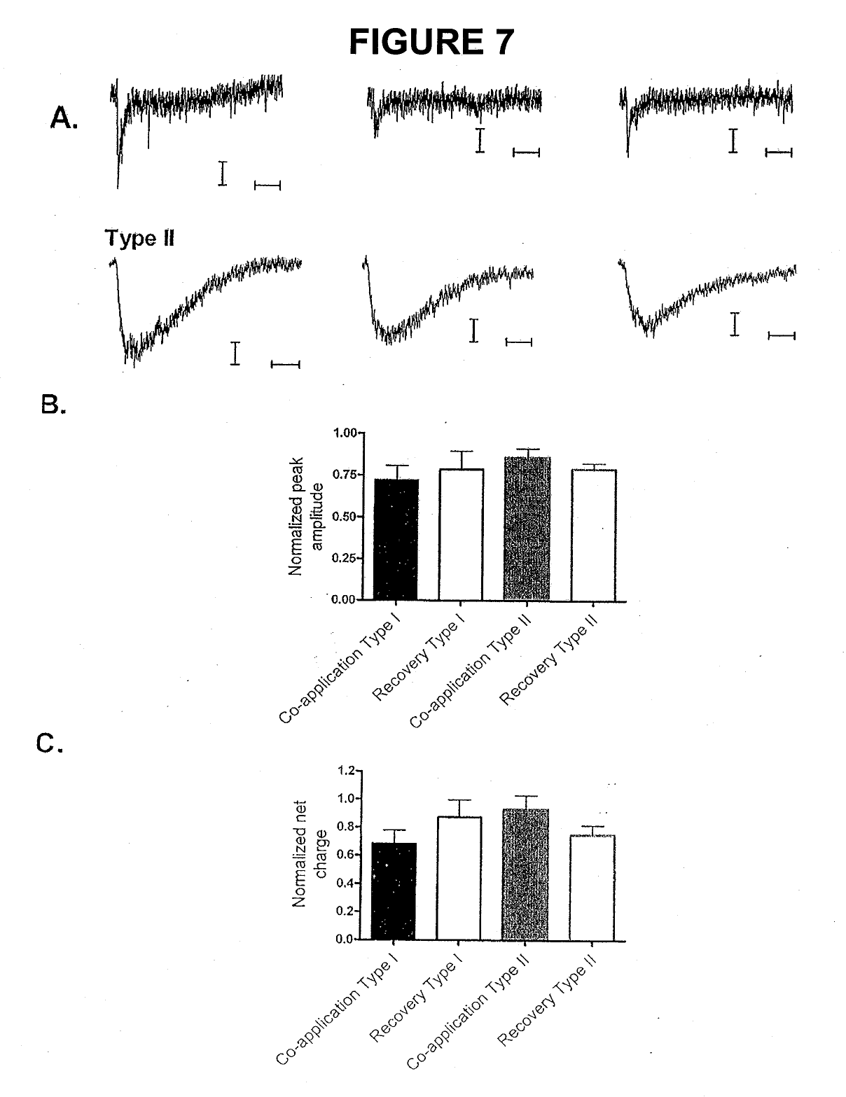

Neurons in rat medial septum vary in their nAChR-mediated responses. Some neurons (Type I cells) have relatively fast transient ACh-evoked responses, while others (Type II cells) have slower ACh-evoked responses (Thinschmidt et al., 2005a). To investigate whether the α7-selectivity of tkP3BzPB would discriminate between these types of ACh responses, a double-barreled picospritzer pressure application system was used with one barrel containing 1 mM ACh and the other containing 1 mM ACh+300 μM tkP3BzPB (FIG. 7). After co-application, Type I cells showed 72±6% and 68±9% of the average baseline peak and net charge response, respectively. On the other hand, Type II cells exhibited 86±5% and 93±10% of the average baseline peak and net charge responses, respectively. In terms of recovery, Type I displayed 79±11% and 87±12% of the average baseline peak and net charge responses, respectively, while the percentages of peak and net charge recovery for Type II were 79±4 and 75±7, respectively. There was no significant difference in the amount of inhibition induced by tkP3BzPB between Type I and Type II cells in medial septum. However this result is not unexpected since tkP3BzPB was only applied briefly in this set of experiments and also match our results obtained from oocytes experiments in which it was observed that inhibition induced by tkP3BzPB was higher when it was pre-applied before the co-application with ACh (FIG. 4D and FIG. 5).

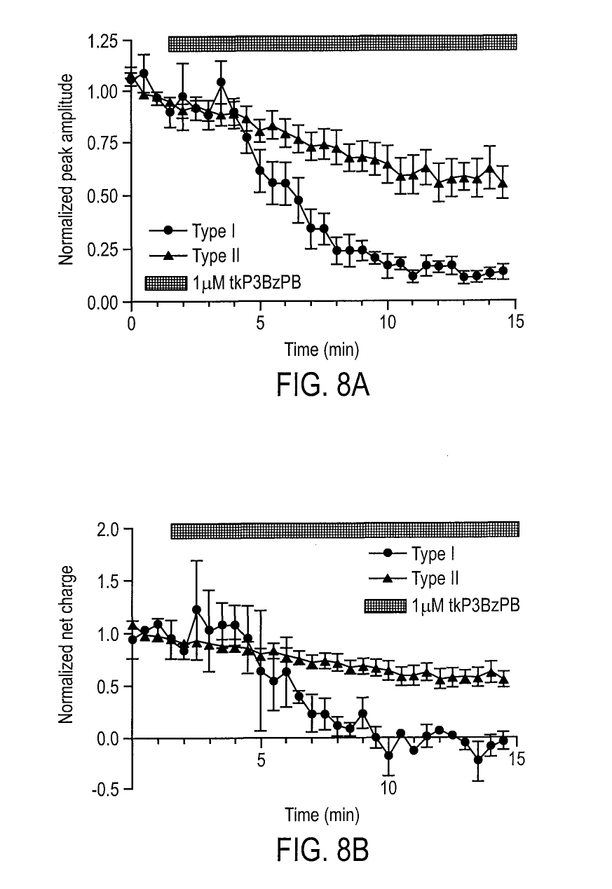

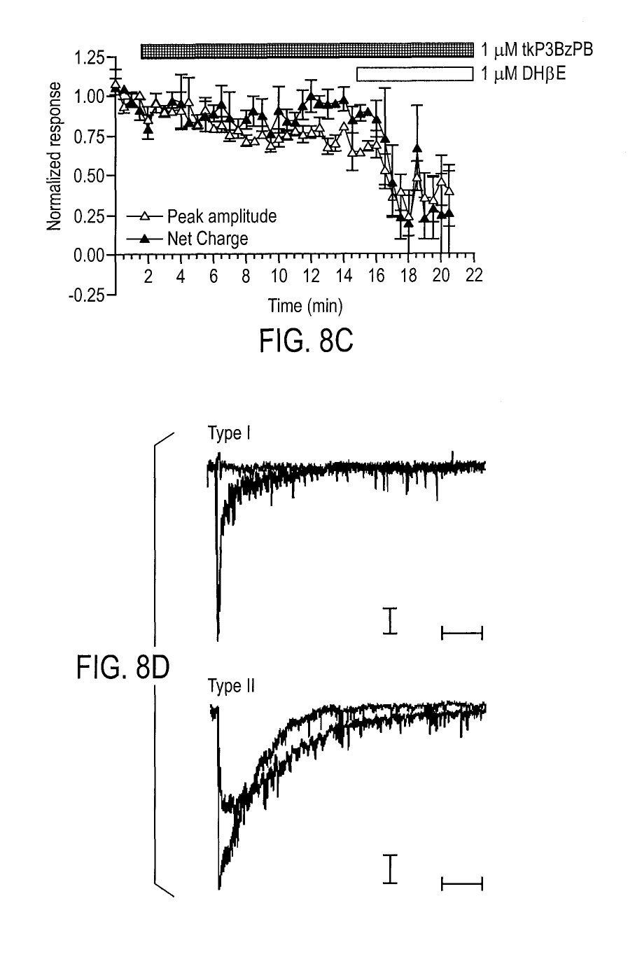

From the oocytes experiments, the IC₅₀ value for tkP3BzPB was higher than that for tPyQB, and further that tkP3BzPB produced a prolonged inhibition of α7 nAChR responses. Based on the oocyte data, prolonged application of tkP3BzPB would be expected to produce a greater inhibition than that produced by repeated application. To test this hypothesis, we conducted experiments in which 1 μM tkP3BzPB was bath-applied to medial septal neurons, similar to experiments performed with hippocampal interneurons (FIG. 8). When evaluating the changes in ACh-evoked peak amplitudes, after tkP3BzPB bath application only 12±0.6% of the initial evoked response remained in Type I cells, while in Type II the percentage of baseline response at the end of the application was 59±1%. This difference between Type I and Type II cells was statistically significant (p<0.001). Regarding net-charge evoked responses after tkP3BzPB bath application, Type II exhibited 79±2% of the baseline response, while Type I responses were fully abolished (p<0.001). As shown in FIG. 8, following bath application of tkP3BzPB, the residual ACh-evoked responses in Type II cells were largely sensitive to DHβE blockade. The difference in the inhibition of α7 nAChR responses by tkP3BzPB between co-application and prolonged bath application experiments in brain slices is consistent with the data obtained in the oocyte expression system (FIG. 6), and supports the hypothesis that tkP3BzPB accumulates over time to produce more inhibition than could be measured with a simple co-application protocol.

TABLE 1
IC₅₀ values for the AQA analogs
IC₅₀ values, μM

| | α7 | α4β2 | α3β4 |
|---|---|---|---|
| tPyQB | 0.13 ± 0.02 | 4.1 ± 1.0 | 1.0 ± 0.1 |
| tPy2PiB | 6.3 ± 0.6 | 103 ± 25 | 10.0 ± 1.4 |
| tkP3BzPB | 1.0 ± 0.1 | 48 ± 11 | 9.2 ± 1.2 |

### Example 2

### Preparation of 1,3,5-tris-(5-hydroxypent-1-ynyl)-benzene

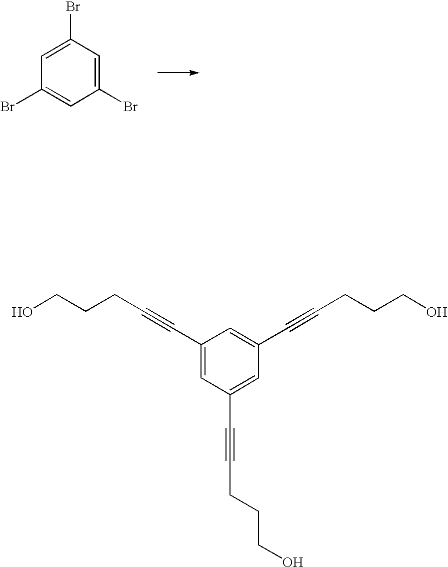

1,3,5-Tribromobenzene (10 g, 31.76 mmol), 4-pentyn-1-ol (10.69 g, 127.06 mmol) and bis(triphenylphosphine)palladium(II) dichloride were stirred in triethylamine under nitrogen for 5 minutes. Copper(I) iodide (92 mg, 0.48 mmol) was added and the mixture was stirred for 6 hours at 80° C. The mixture was cooled to room temperature, filtered through a celite pad and rinsed with ethyl acetate. The combined filtrate was evaporated to dryness under reduced pressure. The resulting residue was purified by column chromatography (CHCl₃:MeOH 10:1) to afford 7.61 g of 1,3,5-tris-(5-hydroxy-1-pentynyl)-benzene. Yield: 74%. ¹H NMR (300 MHz, CDCl₃) δ 7.31 (3, 3 h), 3.81 (t, J=6.0 Hz, 6H), 2.52 (t, J=6.9 Hz, 6H), 1.85 (m, 6H); ¹³C NMR (75 MHz, CDCl₃) δ 133.8, 124.2, 90.5, 80.0, 61.9, 31.5, 16.2 ppm.

### Example 3

### Preparation of 1,3,5-tris-(5-bromopent-1-ynyl)-benzene

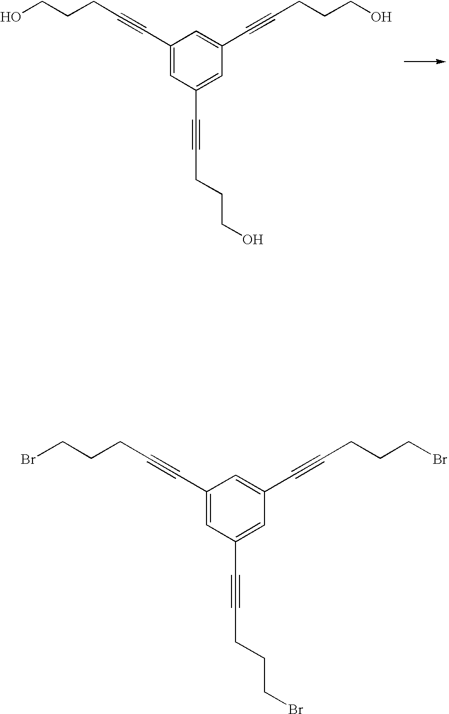

1,3,5-tris-(5-hydroxy-1-pentynyl)-benzene (1.86 g, 5.73 mmol) and carbon tetrabromide (7.41 g, 22.35 mmol) were dissolved in dry methylene chloride (40 mL) and cooled to 0° C. Triphenyl phosphine (6.16 g, 23.47 mmol) was added dropwide and the mixture was stirred at 0° C. for 30 minutes. The mixture was poured into hexanes (200 mL), filtered through a short silica gel column and washed with ethyl acetate/hexanes (1/4). The combined organic solvents were evaporated to dryness under reduced pressure. The resulting residue was purified by column chromatography (hexanes:ethyl acetate 10:1) to afford 2.63 g of 1,3,5-tris-(5-bromopent-1-ynyl)-benzene. Yield 89%. ¹H NMR (300 MHz, CDCl₃) δ 7.33 (s, 3H), 3.57 (t, J=6.3 Hz), 2.60 (t, J=6.9 Hz, 6H), 2.12 (m, 6H) ppm; ¹³C NMR (75 MHz, CDCl₃) δ 133.9, 124.1, 89.2, 80.4, 32.6, 31.7, 18.4 ppm.

### Example 4

### Preparation of 1,3,5-tris-(5-hydroxypentyl)-benzene

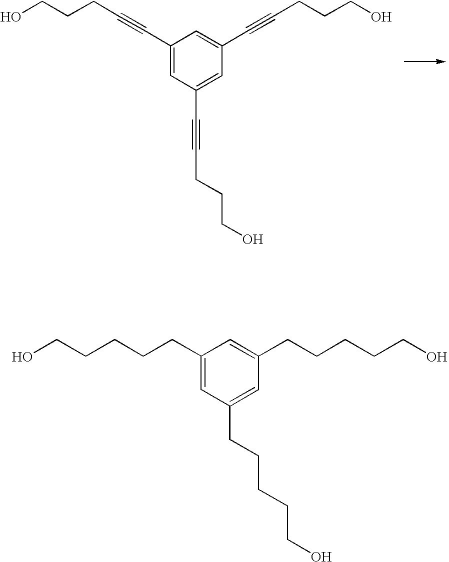

1,3,5-tris-(5-hydroxy-1-pentynyl)-benzene (2.84 g, 8.6 mmol) was dissolved in methanol (30 mL) and 10% Pd/C (5% w/w) was added. The resulting mixture was hydrogenated on a Parr hydrogenation apparatus (45 psi) for 4 hours. The catalyst was removed by filtration through a celite pad. The filter cake was rinsed with methanol, and the combined organic liquors were concentrated under reduced pressure. The crude product was purified by column chromatography (CHCl₃:MeOH 6:1) to afford 2.84 g of 1,3,5-tris-(5-hydroxypentyl)-benzene. Yield 96%. ¹H NMR (300 MHz, CDCl₃) δ 6.81 (s, 3H), 3.62 (t, J=6.3 Hz, 6H), 2.57 (t, J=7.5 Hz, 6H), 1.53-1.70 (m, 12H), 1.38 (m, 6H) ppm; ¹³C NMR (75 MHz, CDCl₃) δ 142.5, 126.1, 63.1, 36.1, 32.9, 31.5, 25.7 ppm.

### Example 5

### Preparation of 1,3,5-tris-(5-bromopentyl)-benzene

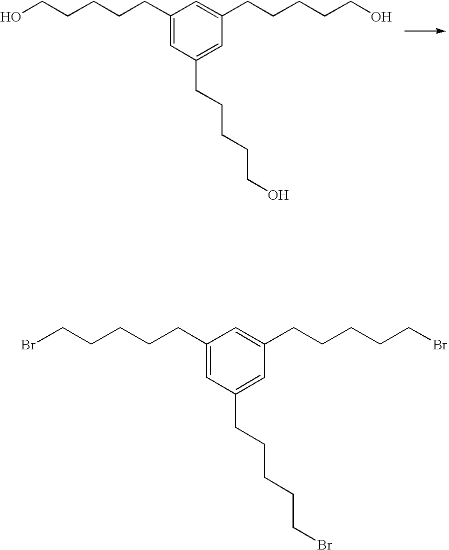

1,3,5-tris-(5-hydroxypentyl)-benzene (2.83 g, 8.41 mmol) and carbon tetrabromide (10.99 g, 32.80 mmol) were dissolved in dry methylene chloride (50 mL) and cooled to 0° C. Triphenyl phosphine (9.03 g, 34.33 mmol) was added dropwise and the mixture was stirred for 30 minutes at 0° C. The mixture was poured into hexanes (250 mL), filtered through a short silica gel column and washed with ethyl acetate/hexanes (1/4). The combined organic solvents were evaporated to dryness under reduced pressure. The resulting residue was purified by column chromatography (hexanes:ethyl acetate 8:1) to afford 4.08 g of 1,3,5-tris-(5-bromopentyl)-benzene. Yield 92%. ¹H NMR (300 MHz, CDCl₃) δ 6.81 (s, 3 h), 3.41 (t, J=6.9 Hz, 6H), 2.60 (t, J=7.5 Hz, 6H), 1.88 (m, 6H), 1.45 (m, 6H) ppm; ¹³C NMR (75 MHz, CDCl₃) δ 142.4, 126.1, 35.9, 34.2, 32.9, 30.9, 28.2 ppm.

### Example 6

### Preparation of 1,3,5-tris-[5-(2-picolinium)-pent-1-ynyl]-benzene tribromide

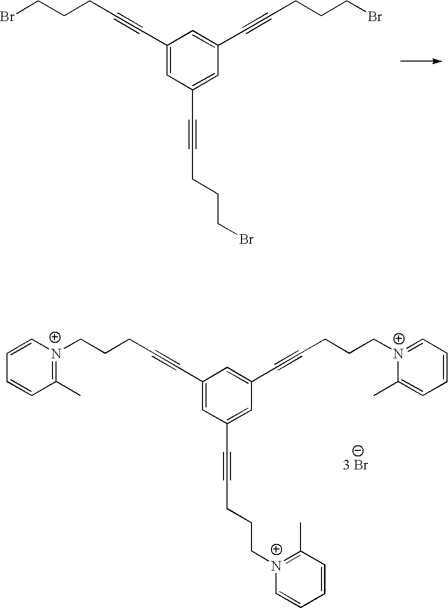

A mixture of 1,3,5-tris-(5-bromopent-1-ynyl)-benzene (223 mg, 0.43 mmol) and 2-picoline (607 mg, 6.52 mmol) was heated at 60-70° C. for 12 hours. The resultant mixture was washed with diethyl ether and then dissolved in water (15 mL), the aqueous solution was washed with diethyl ether, then lyophilized to afford 327 mg of 1,3,5-tris-[5-(2-picolinium)-pent-1-ynyl]-benzene tribromide. Yield 95%. ¹H NMR (300 MHz, CD₃OD) δ 9.01 (dd, J=6.3, 0.9 Hz, 3H), 8.44 (dt, J=7.8, 1.5 Hz, 3H), 7.92-8.07 (m, 6H), 7.41 (s, 3H), 4.81 (t, J=6.0 Hz), 2.98 (s, 9H), 2.70 (t, J=7.2 Hz, 6H), 2.29 (m, 6H) ppm; ¹³C NMR (75 MHz, CD₃OD) δ 157.1, 146.8, 146.6, 134.9, 131.6, 127.1, 125.4, 90.1, 81.6, 58.4, 29.9, 20.7, 17.3 ppm.

### Example 7

### Preparation of 1,3,5-tris-[5-(3-butyl-pyridinium)-pent-1-ynyl]-benzene tribromide

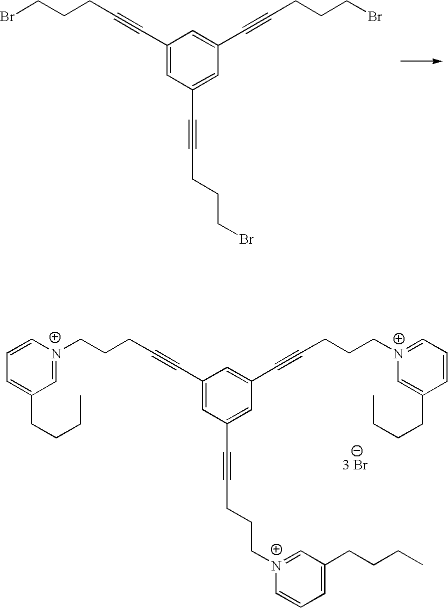

A mixture of 1,3,5-tris-(5-bromopent-1-ynyl)-benzene (240 mg, 0.47 mmol) and 3-butyl pyridine (950 mg, 7.05 mmol) was heated at 60-70° C. for 12 hours. The resultant mixture was washed with diethyl ether and then dissolved in water (15 mL), the aqueous solution was washed with diethyl ether (30 mL×3), then lyophilized to afford 254 mg of 1,3,5-tris-[5-(3-butyl-pyridinium)-pent-1-ynyl]-benzene tribromide. Yield 59%. ¹H NMR (300 MHz, CD₃OD) δ 9.04 (s, 3H), 8.94 (d, J=6.0 Hz, 3H), 8.44 (d, J=8.1 Hz, 3H), 8.04 (dd, J=8.1, 6.0 Hz, 3H), 7.33 (s, 3H), 4.82 (t, J=7.2 Hz, 6H), 2.87 (t, J=7.8 Hz, 6H), 2.63 (t, J=6.9 Hz, 6H), 2.35 (m, 6H), 1.69 (m, 6H), 1.42 (m, 6H), 0.97 (t, J=7.5 Hz, 9H) ppm; ¹³C NMR (75 MHz, CD₃OD) δ 146.8, 145.8, 145.6, 143.6, 135.0, 129.0, 125.3, 89.9, 81.5, 62.2, 33.8, 33.5, 30.9, 23.5, 17.2, 14.3 ppm.

### Example 8

### Preparation of 1,3,5-tris-[5-(3-phenyl-pyridinium)-pent-1-ynyl]-benzene tribromide

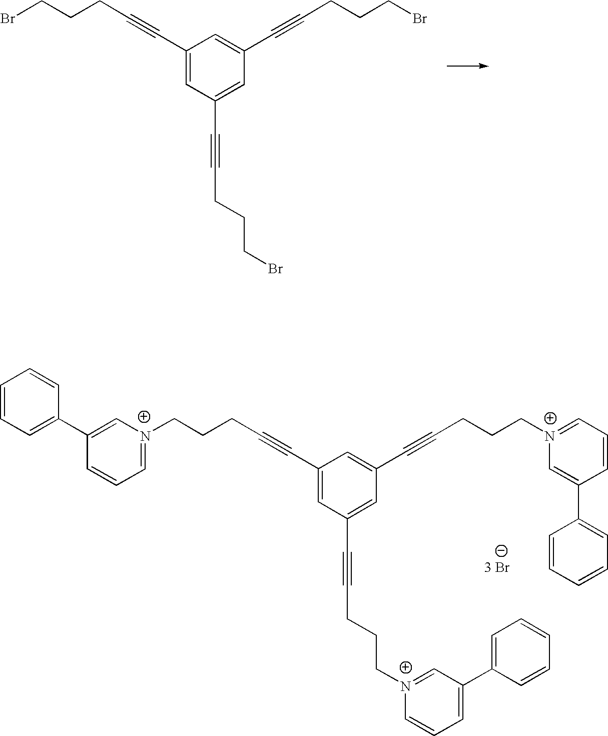

A mixture of 1,3,5-tris-(5-bromopent-1-ynyl)-benzene (259 mg, 0.50 mmol) and 3-phenyl pyridine (1.18 g, 7.50 mmol) was heated at 60-70° C. for 12 hours. The resultant mixture was washed with diethyl ether and then dissolved in water (15 mL), the aqueous solution was washed with diethyl ether (30 mL×5), then lyophilized to afford 415 mg of 1,3,5-tris-[5-(3-phenyl-pyridinium)-pent-1-ynyl]-benzene tribromide. Yield 85%. ¹H NMR (300 MHz, CD₃OD) δ 9.50 (s, 3H), 9.09 (d, J=6.0 Hz, 3H), 8.78 (d, J=8.1 Hz, 3H), 8.16 (dd, J=8.1, 6.0 Hz, 3H), 7.75-7.87 (m, 6H), 7.42-7.65 (m, 9H), 7.17 (s, 3H), 4.96 (t, J=6.9 Hz, 6H), 2.69 (t, J=6.3 Hz, 6H), 2.42 (m, 6H) ppm; ¹³C NMR (75 MHz, CD₃OD) δ 144.4, 144.3, 144.2, 142.6, 134.9, 134.5, 131.5, 130.8, 129.5, 128.7, 125.1, 90.0, 81.5, 62.6, 30.8, 17.4 ppm.

### Example 9

### Preparation of 1,3,5-tris-{5-[3-(1-methyl-2-S-pyrrolidinyl)pyridinium]-pent-1-ynyl}-benzene tribromide

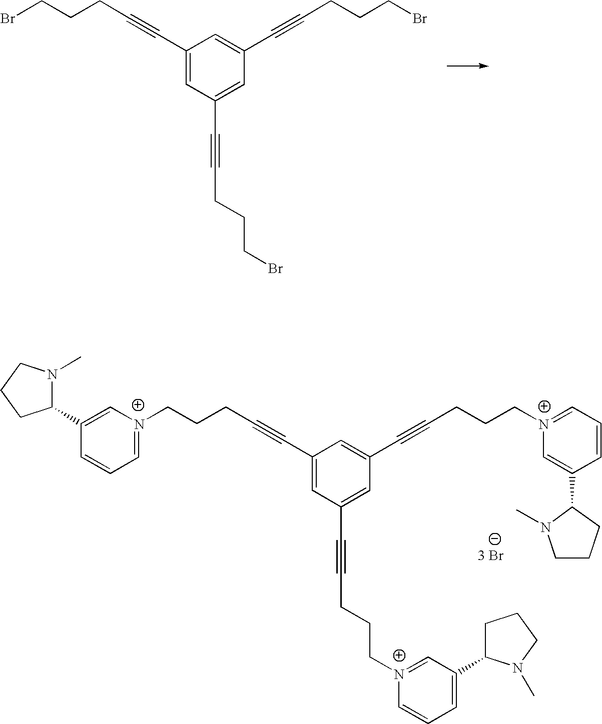

A mixture of 1,3,5-tris-(5-bromopent-1-ynyl)-benzene (246 mg, 0.48 mmol) and S-nicotine (1.5 mL) was heated at 60-70° C. for 12 hours. The resultant mixture was washed with diethyl ether and then dissolved in water (15 mL), the aqueous solution was washed with diethyl ether (30 mL×5), then lyophilized to afford 440 mg of 1,3,5-tris-{5-[3-(1-methyl-2-S-pyrrolidinyl)pyridinium]-pent-1-ynyl}-benzene tribromide. Yield 92%. ¹H NMR (300 MHz, CD₃OD) δ 9.16 (s, 3H), 9.02 (d, J=6.0 Hz, 3H), 8.60 (d, j=8.1 Hz, 3H), 8.12 (dd, J=8.1, 6.0 Hz, 3H), 7.40 (s, 3H), 4.86 (t, J=6.9 Hz, 6H), 3.69 (m, 3H), 3.32 (m, 6H), 2.14-2.70 (m, 12H), 2.33 (s, 9H), 1.73-2.14 (m, 10H) ppm; ¹³C NMR (75 MHz, CD₃OD) δ 146.2, 145.4, 145.0, 135.1, 129.5, 125.3, 89.8, 81.8, 68.8, 62.3, 58.0, 40.8, 36.0, 30.8, 24.1, 17.1 ppm.

### Example 10

### Preparation of 1,3,5-tris-[5-(1-quinolinium)-pent-1-ynyl]-benzene tribromide

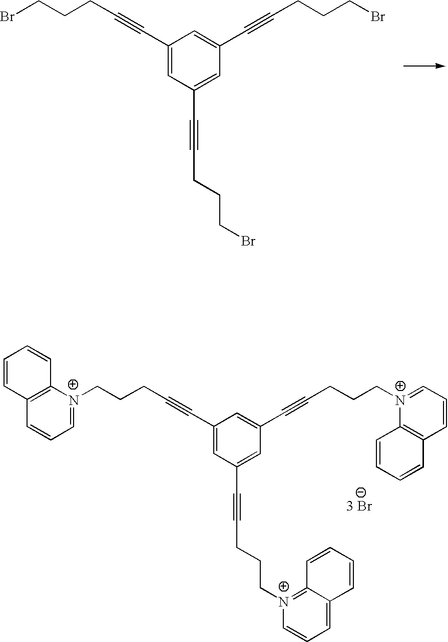

A mixture of 1,3,5-tris-(5-bromopent-1-ynyl)-benzene (232 mg, 0.45 mmol) and quinoline (880 mg, 6.75 mmol) was heated at 60-70° C. for 12 hours. The resultant mixture was washed with diethyl ether and then dissolved in water (15 mL), the aqueous solution was washed with diethyl ether (30 mL×5), then lyophilized to afford 234 mg of 1,3,5-tris-[5-(1-quinolinium)-pent-1-ynyl]-benzene tribromide. Yield 58%. ¹H NMR (300 MHz, CD₃OD) δ 9.58 (dd, J=6.0, 4.5 Hz, 3H), 9.18 (d, J=8.1 Hz, 3H), 8.67, d, J=9.0 Hz, 3H), 8.41 (dd, J=8.1, 1.8 Hz, 3H), 8.31 (m, 3H), 7.98-8.18 (m, 6H), 7.11 (s, 3H), 5.32 (t, J=6.9 Hz, 6H), 2.75 (t, J=6.6 Hz, 6H), 2.46 (m, 6H) ppm; ¹³C NMR (75 MHz, CD₃OD) δ 150.9, 149.3, 139.6, 137.4, 134.8, 132.3, 131.8, 131.4, 125.2, 123.2, 119.8, 90.2, 81.4, 58.8, 29.6, 17.5 ppm.

### Example 11

### Preparation of 1,3,5-tris-[5-(2-isoquinolinium)-pent-1-ynyl]-benzene tribromide

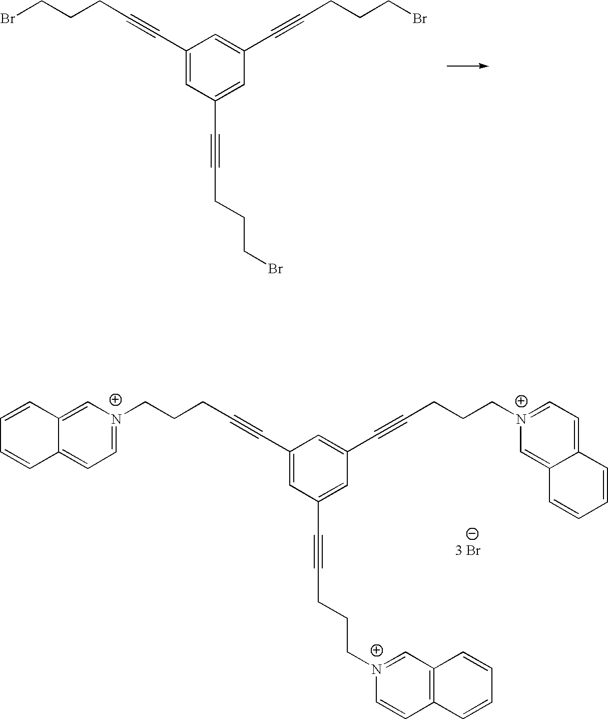

A mixture of 1,3,5-tris-(5-bromopent-1-ynyl)-benzene (222 mg, 0.43 mmol) and isoquinoline (840 mg, 6.45 mmol) was heated at 60-70° C. for 12 hours. The resultant mixture was washed with diethyl ether and then dissolved in water (15 mL), the aqueous solution was washed with diethyl ether (30 mL×5), then lyophilized to afford 357 mg of 1,3,5-tris-[5-(2-isoquinolinium)-pent-1-ynyl]-benzene tribromide. Yield 92%. ¹H NMR (300 MHz, CD₃OD) δ 10.15 (s, 3H), 8.79 (dd, J=6.9, 1.2 Hz, 3H), 8.67, d, J=9.0 Hz, 3H), 8.50 (m, 3H), 8.20 (m, 6H), 8.06 (m, 3H), 6.54 (s, 3H), 5.00 (t, J=6.9 Hz, 6H), 2.75 (t, J=6.3 Hz, 6H), 2.47 (m, 6H) ppm; ¹³C NMR (75 MHz, CD₃OD) δ 151.5, 139.2, 138.5, 136.0, 134.1, 132.6, 131.6, 129.2, 128.4, 127.5, 124.7, 90.0, 81.2, 62.6, 30.5, 17.6 ppm.

### Example 12

### Preparation of 1,3,5-tris-[5-(3-phenyl-pyridinium)-pentyl]-benzene tribromide

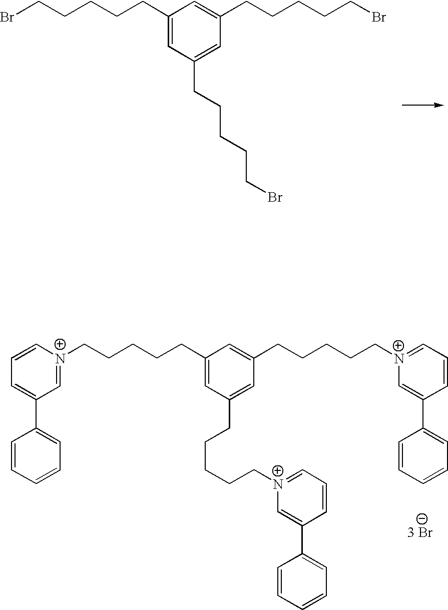

A mixture of 1,3,5-tris-(5-bromopentyl)-benzene (272 mg, 0.52 mmol) and 3-phenyl-pyridine (323 mg, 2.08 mmol) was dissolved in butanone (5 mL) and heated at reflux for 24 hours. The resultant mixture was washed with diethyl ether and then dissoved in water (15 mL), the aqueous solution was washed with diethyl ether (30 mL×5), then lyophilized to afford 215 mg of 1,3,5-tris-[5-(3-butyl-pyridinium)-pentyl]-benzene tribromide. Yield: 42%. ¹H NMR (300 MHz, CD₃OD) δ 9.39 (s, 3H), 8.98 (d, J=6.0 Hz, 3H), 8.85 (ddd, J=6.0, 1.8, 1.2 Hz, 3H), 8.15 (dd, J=8.1, 6.0 Hz, 3H), 7.78-7.90 (m, 6H), 7.50-7.65 (m, 9H), 6.82 (s, 3H), 4.74 (t, 7.8 Hz, 6H), 2.55 (t, J=7.6 Hz, 6H), 2.11 (m, 6H), 1.69 (m, 6H), 1.45 (m, 6H) ppm; ¹³C NMR (75 MHz, CD₃OD) δ 144.2, 143.9, 143.4, 142.8, 134.6, 131.5, 130.8, 129.4, 128.7, 127.2, 63.3, 36.7, 32.7, 32.2, 27.0 ppm.

### Example 13

### Preparation of 1,3,5-tris-{5-[3-(1-methyl-2-S-pyrrolidinyl)pyridinium]-pentyl}-benzene tribromide

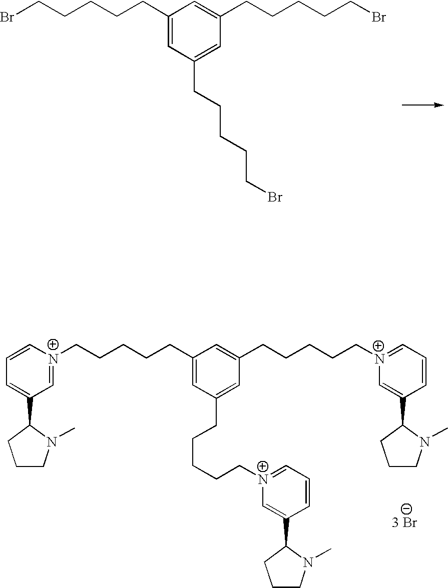

A mixture of 1,3,5-tris-(5-bromopentyl)-benzene (297 mg, 0.57 mmol) and S-nicotine (1.5 mL) was heated at 60-70° C. for 12 hours. The resultant mixture was washed with diethyl ether and then dissolved in water (15 mL), the aqueous solution was washed with diethyl ether (30 mL×5), then lyophilized to afford 510 mg of 1,3,5-tris-{5-[3-(1-methyl-2-S-pyrrolidinyl)pyridinium]-pentyl}-benzene tribromide. Yield 89%. ¹H NMR (300 MHz, CD₃OD) δ 9.08 (s, 3H), 8.94 (d, J=6.0 Hz, 3H), 8.61 (d, J=8.1 Hz, 3H), 8.08 (dd, J=8.1, 6.0 Hz, 3H), 6.83 (s, 3H), 4.67 (t, J=7.5 Hz, 6H), 3.68 (t, 7.5 Hz, 3H), 3.37 (m, 6H), 2.35-2.65 (m, 12H), 2.32 (s, 9H), 1.75-2.17 (m, 12H), 1.69 (m, 6H), 1.43 (m, 6H) ppm; ¹³C NMR (75 MHz, CD₃OD) δ 145.8, 145.1, 144.8, 143.4, 129.4, 127.2, 68.8, 63.1, 58.0, 40.7, 36.7, 36.0, 32.6, 32.2, 27.0, 24.0 ppm.

### Example 14

### Preparation of 1,3,5-tris-[5-(1-quinolinium)-pentyl]-benzene tribromide

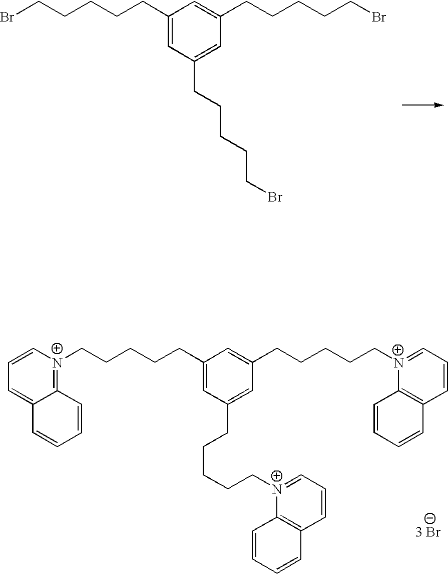

A mixture of 1,3,5-tris-(5-bromopentyl)-benzene (251 mg, 0.48 mmol) and quinoline (930 mg, 7.20 mmol) was heated at 60-70° C. for 12 hours. The resultant mixture was washed with diethyl ether and then dissolved in water (15 mL), the aqueous solution was washed with diethyl ether (30 mL×5), then lyophilized to afford 390 mg of 1,3,5-tris-[5-(1-quinolinium)-pentyl]-benzene tribromide. Yield 89%. ¹H NMR (300 MHz, CD₃OD) δ 9.46 (dd, J=6.0, 1.5 Hz, 3H), 9.22 (d, J=8.4 Hz, 3H), 8.57 (d, J=9.0 Hz, 3H), 8.45 (dd, J=8.4, 1.5 Hz, 3H), 8.30 (m, 3H), 8.02-8.14 (m, 6H), 6.8 (s, 3H), 5.11 (t, 7.5 Hz, 6H), 2.56 (t, J=7.5 Hz, 6H), 2.14 (m, 6H), 1.69 (m, 6H), 1.52 (m, 6H) ppm; ¹³C NMR (75 MHz, CD₃OD) δ 150.3, 148.9, 143.4, 139.4, 137.3, 132.2, 131.8, 131.4, 127.2, 123.1, 119.9, 59.4, 36.7, 32.3, 31.1, 27.3 ppm.

### Example 15

### Preparation of 1,3,5-tris-[5-(2-isoquinolinium)-pentyl]-benzene tribromide

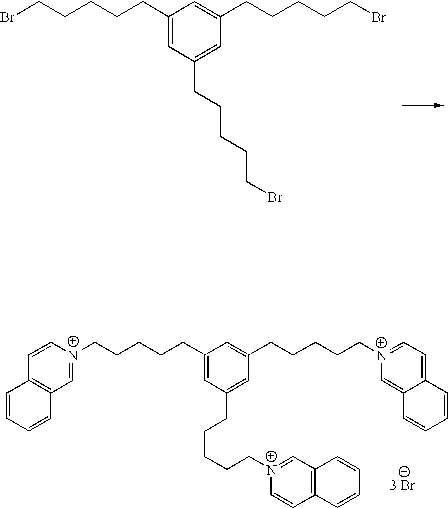

A mixture of 1,3,5-tris-(5-bromopentyl)-benzene (266 mg, 0.51 mmol) and quinoline (988 mg, 7.65 mmol) was heated at 60-70° C. for 12 hours. The resultant mixture was washed with diethyl ether and then dissolved in water (15 mL), the aqueous solution was washed with diethyl ether (30 mL×5), then lyophilized to afford 410 mg of 1,3,5-tris-[5-(2-isoquinolinium)-pentyl]-benzene tribromide. Yield 89%. ¹H NMR (300 MHz, CD₃OD) δ 9.99 (s, 3H), 8.69 (dd, J=6.9, 1.5 Hz, 3H), 8.47-8.54 (m, 6H), 8.22-8.36 (m, 6H), 8.07 (m, 3H), 4.78 (t, J=7.5 Hz, 6H), 2.53 (t, J=7.5 Hz, 6H), 2.15 (m, 6H), 1.67 (m, 6H), 1.44 (m, 6H); ¹³C NMR (75 MHz, CD₃OD) δ 150.8, 143.3, 138.8, 138.2, 135.8, 132.5, 131.5, 129.0, 128.5, 127.5, 127.1, 62.8, 36.5, 32.3, 32.0, 26.8 ppm.

### Example 16

### Preparation of 1,2,4,5-tetraiodobenzene

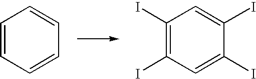

Periodic acid (2.56 g, 11.2 mmol) was dissolved with stirring in concentrated H₂SO₄ (60 mL) Potassium iodide (5.58 g, 33.6 mmol) was crushed and added to the clear solution. After about 30 min of stirring, the dark mixture was placed in an ice bath. The aromatic substrate (C₆H₅, 1 mL, 11.2 mmol) was then added slowly. The reaction was allowed to stir to room temperature for 1 day and poured onto crushed ice. The resulting solid was collected by suction filtration and washed well with methanol to remove iodine. The crude lavender powder (5.4 g 82% yield) was crystallized from 2-methoxyethanol, giving 1,2,4,5-tetraiodobenzene (71% yield) as white needles, mp 252-255° C. ¹H NMR (Me₂SO-d6) δ 8.32 (s); ¹³C NMR (Me₂SO-d6) 147.1, 108.5 ppm.

### Example 17

### Preparation of 5,5′,5″,5′″-(1,2,4,5-benzentetrayl)-tetrakis(4-pentyn-ol)

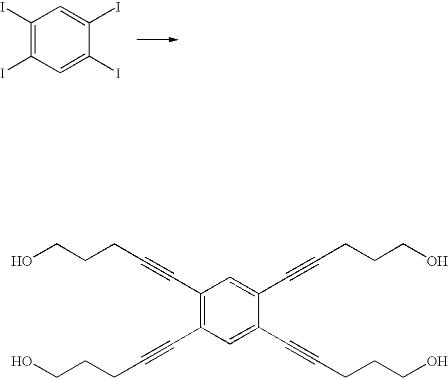

To a degassed solution of 1,2,4,5-tetraiodobenzene (5.81 g, 0.01 mol) in DMF-Et₃N (100 mL, 1:1) were added Pd(PPh₃)₂Cl₂ (350 mg. 0.5 mmol), CuI (200 mg, 1.2 mmol), and 4-pentyn-1-ol (4.2 g, 0.05 mol) was added drop-wise. The mixture was stirred under N₂ at room temperature for 24 h. The solution was poured into water (400 mL). The mixture was extracted with CH₂Cl₂ (3×200 mL). The combined organic phases were washed with 5% HCl and brine, dried over Na₂SO₄, and concentrated under vacuum. The residue was purified by silica gel column chromatography using CH₂Cl₂MeOH (10:1, v/v) as eluent to afford tetramer (3.34 g, 82%): ¹H NMR (300 MHz, CD₃Cl+CO₃OD δ ppm), 7.30 (s, 2H), 3.72 (t, J=6.3 Hz, 8H), 2.52 (t, J=7.2 Hz, 8H), 1.80 (p, J=6.6 Hz, 8H) ppm.

### Example 18

### Preparation of 5,5′,5″,5′″-(1,2,4,5-benzentetrayl)-tetrakis(1-bromo-4-pentyne)

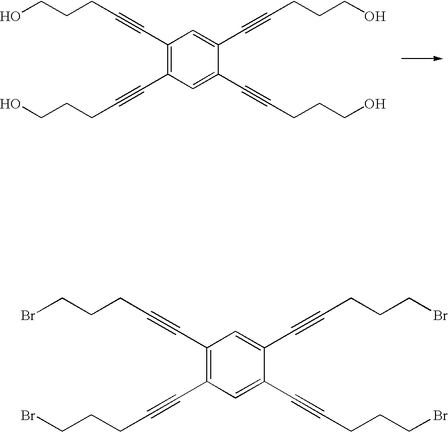

5,5′,5″,5′″-(1,2,4,5-benzentetrayl)tetrakis-4-pentyn-ol (2.05 g, 5.03 mmol) and carbon tetrabromide (7.41 g, 22.35 mmol) were dissolved in dry methylene chloride (100 mL) and cooled to 0° C. Triphenyl phosphine (6.16 g, 23.47 mmol) was added portion-wise and the mixture was stirred at RT. After the starting alcohol was consumed methanol was added and the mixture was stirred for an additional 5 minutes. The mixture concentrated and was treated with hexanes (500 mL) and then filtered through a short silica gel column, washed with ethylacetate/hexanes (1/4). The combined organic solvents were evaporated to dryness under reduced pressure. The resulting residue was purified by column chromatography (hexanes) to afford 2.84 g of the title compound. Yield: 89%. ¹H NMR (300 MHz, CDCl₃) δ 7.39 (s, 2H), 3.62 (t, J=6.3 Hz, 8H), 2.67 (t, J=6.6 Hz, 8H), 1.14 (p, J=6.6 Hz, 8H) ppm.

### Example 19

### Preparation of 5,5′,5″,5′″-(1,2,4,5-benzentetrayl)-tetrakis-(pentan-1-ol)

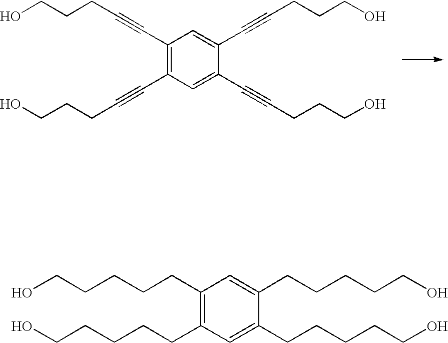

5,5′,5″,5′″-(1,2,4,5-benzentetrayl)tetrakis-4-pentyn-1-ol (2.01 g, 4.95 mmol) was dissolved in methanol (30 mL) and 10% Pd/C (5% w/w) was added. The resulting mixture was hydrogenated on a Parr hydrogenation apparatus (45 psig) for 4 hrs. The catalyst was removed by filtration through a Celite pad. The filter cake was rinsed with methanol, and the combined organic liquors were concentrated under reduced pressure. The crude product was purified by column chromatography (CHCl₃:MeOH, 10:1) to afford 1.97 g of the title compound. Yield: 95%. ¹H NMR (300 MHz, CDCl₃) δ 6.81 (s, 3H), 3.62 (t, J=6.3 Hz, 6H), 2.57 (t, J=7.5 Hz, 6H), 1.53-1.70 (m, 12H), 1.38 (m, 6H) ppm; ¹³C NMR (75 MHz, CDCl₃) δ 142.5, 126.1, 63.1, 36.1, 32.9, 31.5, 25.7 ppm.

### Example 20

### Preparation of 5′,5″,5′″-(1,2,4,5-benzentetrayl)-tetrakis(1-bromopentane)

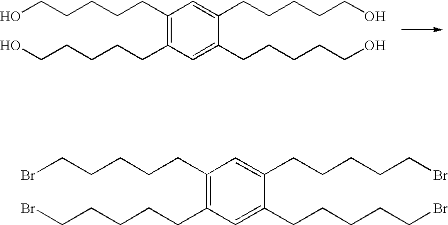

5,5′,5″,5′″-(1,2,4,5-benzentetrayl)-tetrakis-pentan-1-ol (1.97 g, 4.67 mmol) and carbon tetrabromide (7.22 g, 21.74.80 mmol) were dissolved in dry methylene chloride (50 mL) and cooled to 0° C. Triphenyl phosphine (5.70 g, 22.02 mmol) was added portion-wise and the mixture was stirred at RT. After the starting alcohol was consumed methanol was added and the mixture was stirred for an additional 5 minutes. The mixture concentrated and was treated with hexanes (500 mL) and then filtered through a short silica gel column, and washed with ethylacetate/hexanes (1/4). The combined organic solvents were evaporated to dryness under reduced pressure. The resulting residue was purified by column chromatography (hexanes) to afford 2.83 g of the title compound. Yield: 90%. ¹H NMR (300 MHz, CDCl₃) δ 6.89 (s, 2H), 3.42 (t, J=7.2 Hz, 8H), 2.55 (m, 8H), 1.90 (m, 8H), 1.45-1.62 (m, 16H) ppm.

### Example 21

### Preparation of 5,5′,5″,5′″-(1,2,4,5-benzentetrayl)-tetrakis(4-pentyn-1-yl-nicotinium) tetrabromide

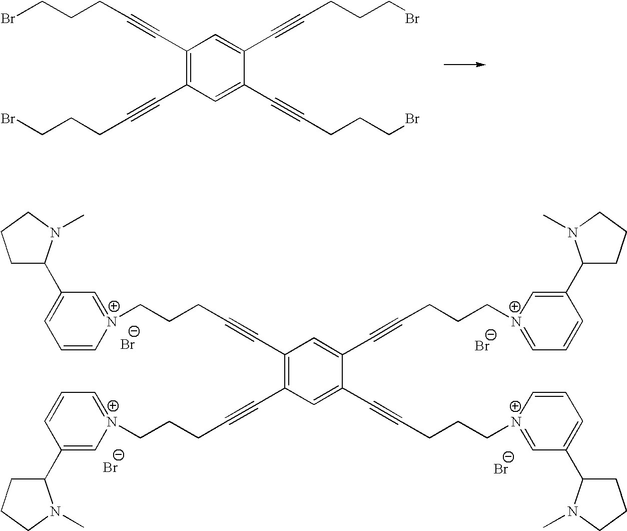

A mixture of 5,5′,5″,5′″-(1,2,4,5-benzentetrayl)tetrakis-1-bromo-4-pentyne (300 mg, 0.46 mmol) and S(−)-nicotine (320 mg, 2 mmol) in acetonitrile was heated at 60-70° C. for 24 hrs. The resulted mixture was treated with diethyl ether and then dissolved in water (15 mL), the aqueous solution was extracted extensively with chloroform (30 mL×5). Water was removed by lyophilization to afford 390 mg of the title compound. Yield: 60%. ¹H NMR (300 MHz, CD₃OD) δ 99.15 (s, 4H), 9.05 (d, J=6 Hz, 4H), 8.54 (d, J=7.8 Hz, 4H), 8.10 (t, J1=6 Hz, J2=7.8 Hz, 4H), 7.48 (s, 2H), 4.91 (t, J=7.5 Hz, 8H), 3.55 (t, J=8.1 Hz, 4H), 3.24 (m, 4H), 2.71 (t, J=8.1 Hz, 8H), 2.36-2.47 (m, 16H), 2.25 (s, 12H), 1.89-1.95 (m, 8H), 1.72-1.76 (m, 4H) ppm.

### Example 22

### Preparation of 5,5′,5″, 5′″-(1,2,4,5-benzentetrayl)-tetrakis[4-pentyn-1-yl-(5,6,7,8-tetrahydroisoquinolinium)]tetrabromide

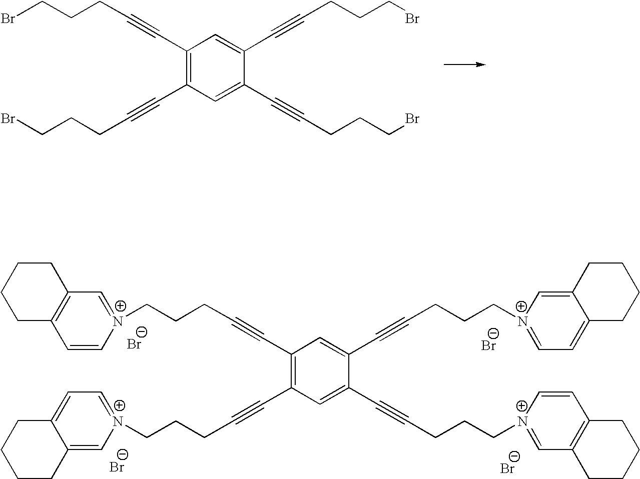

A mixture of 5,5′,5″,5′″-(1,2,4,5-benzentetrayl)tetrakis-1-bromo-4-pentyne (300 mg, 0.46 mmol) and 5,6,7,8-tetrahydroisoquinoline (260 mg, 2.0 mmol) was heated at 60-70° C. for 18 hrs. The resulted mixture was treated with diethyl ether and then dissolved in water (15 mL), the aqueous solution was extracted extensively with chloroform (30 mL×5). Water was removed by lyophilization to afford 320 mg of the title compound. Yield: 80%. ¹H NMR (300 MHz,) δ (CD₃Cl), 8.86 (s, 4H), 8.72 (d, 4H), 7.75 (s, 4H), 7.30 (s, 2H), 4.74 (t, J=8.1 Hz, 8H), 2.91 (br, 16H), 2.72 (t, J=6.6 Hz, 8H), 2.35 (m, 8H), 1.80 (br, 16H). ¹³C NMR, 159.92, 145.40, 141.74, 140.03, 136.20, 129.26, 126.28, 95.18, 80.49, 61.50, 30.94, 30.56, 27.51, 22.30, 22.19, 17.73 ppm.

### Example 23

### Preparation of 5,5′,5″,5′″-(1,2,4,5-benzentetrayl)-tetrakis[4-pentyn-1-yl-(3-phenyl-pyridinium)]tetrabromide

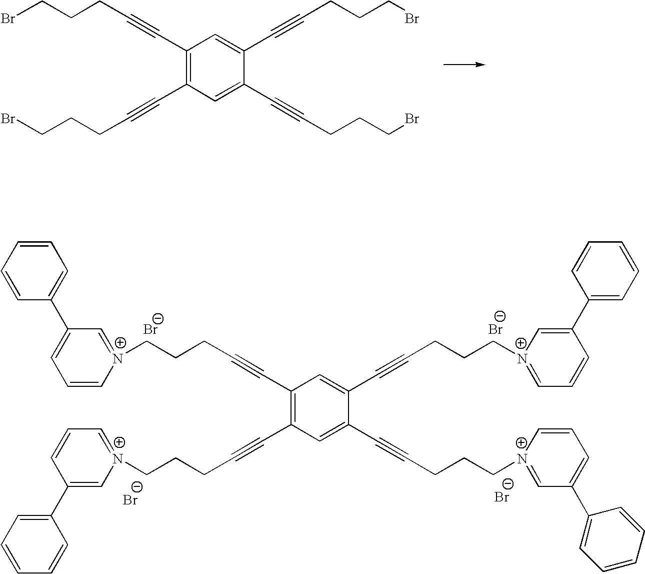

A mixture of 5,5′,5″,5′″-(1,2,4,5-benzentetrayl)tetrakis-1-bromo-4-pentyne (300 mg, 0.46 mmol) and 3-phenylpyridine (310 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 440 mg of the title compound. Yield: 75%. ¹H NMR (300 MHz, CD₃OD) δ ppm 9.50 (s, 4H), 9.09 (d, J=6.0 Hz, 4H), 8.73-8.76 (m, 4H), 7.12-7.16 (m, 4H), 7.76-7.81 (m, 8H), 7.52-7.57 (m, 12H), 7.23 (s, 2H), 4.95 (t, J=8.4 Hz, 8H), 2.71 (t, J=6.6 Hz, 8H), 2.40 (m, 8H) ppm. ¹³C NMR, 144.23, 142.61, 136.23, 134.47, 131.51, 130.78, 139.56, 128.70, 126.13, 94.82, 80.97, 62.58, 31.08, 17.68 ppm.

### Example 24

### Preparation of 5,5′,5″, 5′″-(1,2,4,5-benzentetrayl)-tetrakis[4-pentyne-1-yl-(isoquinolinolinium)]tetrabromide

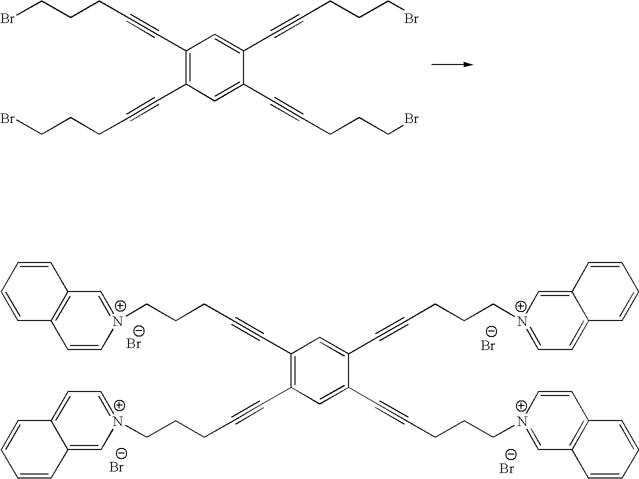

A mixture of 5,5′,5″,5′″-(1,2,4,5-benzentetrayl)tetrakis-1-bromo-4-pentyne (300 mg, 0.46 mmol) and isoquinoline (260 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 442 mg of the title compound. Yield: 82%. NMR (300 MHz, CD₃OD) δ 10.14 (s, 4H), 8.80 (d, J=6.6 Hz, 4H), 8.47 (d, J=7.5 Hz, *H), 8.147-8.17 (m, 8H), 8.00 (m, 4H), 6.59 (s, 2H), 5.02 (t, J=6.6 Hz, 8H), 2.79 (t, J=6.3 Hz, 8H), 2.44-2.50 (m, 8H) ppm. ¹³C NMR, 144.23, 142.61, 136.23, 134.48, 131.51, 130.78, 129.56, 128.70, 126.13, 94.82, 80.97, 62.57, 31.08, 17.68 ppm.

### Example 25

### Preparation of 5,5′,5″,5′″-(1,2,4,5-benzentetrayl)-tetrakis[4-pentyn-1-yl-(3-benzyl-pyridinium)]tetrabromide

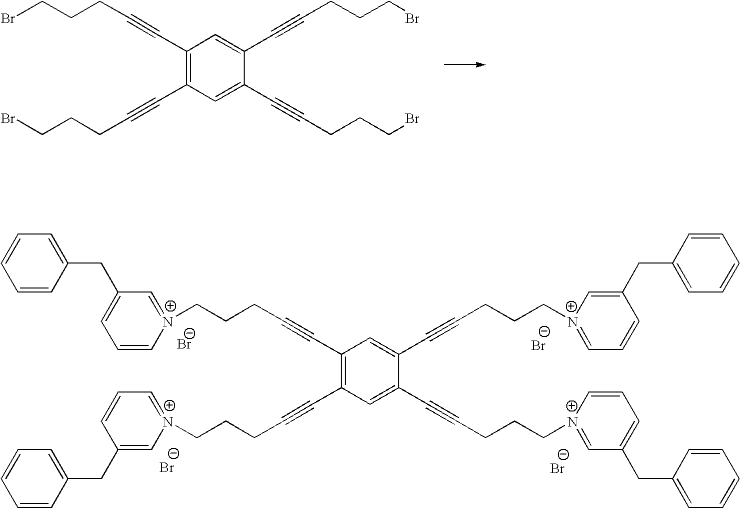

A mixture of 5,5′,5″,5′″-(1,2,4,5-benzentetrayl)tetrakis-1-bromo-4-pentyne (300 mg, 0.46 mmol) and 3-benzyl pyridine (340 mg, 2.0 mmol) was heated at 60-70° C. for 18 hrs. The resulting mixture was treated with diethyl ether and then dissolved in water (15 mL), the aqueous solution was extracted extensively with chloroform (30 mL×5). Water was removed by lyophilization to afford 521 mg of the title compound. Yield: 85%. ¹H NMR (300 MHz, CD₃OD) δ 9.21 (s, 4H), 9.02 (d, 4H), 8.26 (d, 4H), 8.00 (dd, 4H), 7.42 (s, 2H), 7.18-7.28 (m, 20H), 4.86 (t, 8H), 4.22 (s, 8H), 2.67 (t, 8H), 2.34-2.44 (m, 8H) ppm.

### Example 26

### Preparation of 5,5′,5″,5′″-(1,2,4,5-benzentetrayl)-tetrakis[pentanyl-(isoquinolinium)]tetrabromide

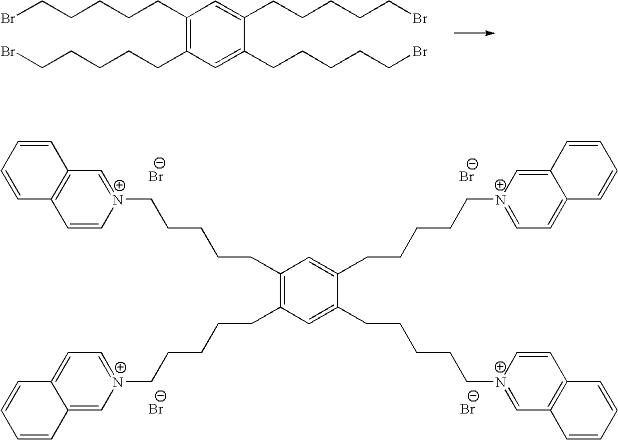

A mixture of 5,5′,5″,5′″-(1,2,4,5-benzentetrayl)-tetrakis-[1-bromopentane] (330 mg, 0.49 mmol) and 3-(3-hydroxypropanyl)-pyridine (300 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 460 mg of the title compound. Yield: 79%. NMR (300 MHz, CD₃OD) δ 10.15 (s, 4H), 8.78-8.81 (m, 4H), 8.49-8.54 (m, 8H), 8.27-8.32 (m, 4H), 8.17-8.28 (m, 4H), 8.00-8.05 (m, 4H), 6.80 (s, 2H), 4.87 (t, J=7.5 Hz, 8H), 2.44-2.51 (m, 8H), 2.15-2.22 (m, 8H), 1.46-1.58 (m, 16H) ppm. ¹³C NMR 149.62, 137.62, 137.18, 137.12, 134.77, 131.40, 130.42, 130.07, 127.84, 127.39, 126.38, 61.72, 31.97, 31.38, 30.94, 26.23 ppm.

### Example 27

### Preparation of 5,5′,5″,5′″-(1,2,4,5-benzentetrayl)-tetrakis[pentanyl-(3-benzylpyridinium)]tetrabromide

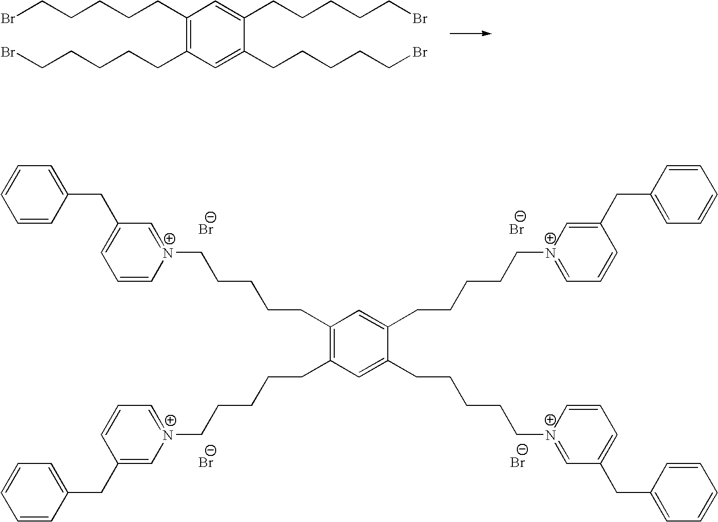

A mixture of 5,5′,5″,5′″-(1,2,4,5-benzentetrayl)tetrakis-[1-bromopentane] (330 mg, 0.49 mmol) and 3-benzyl-pyridine (390 mg, 2.3 mmol) was heated at 60-70° C. for 18 hrs. The resulting mixture was treated with diethyl ether and then dissolved in water (15 mL), the aqueous solution was extracted extensively with chloroform (30 mL×5). Water was removed through lyophilization to afford 575 mg of the title compound. Yield: 87%. ¹H NMR (300 MHz, CD₃OD) δ 9.20 (s, 4H), 8.97 (d, J=6.0 Hz, 4H), 8.41 (d, J=8.1 Hz, 4H), 8.00 (dd, 4H), 7.19-7.34 (m, 20H), 6.91 (s, 2H), 4.70 (t, J=7.5 Hz, 8H), 4.27 (s, 8H), 2.51-2.57 (m, 8H), 2.01-2.11 (m, 8H), 1.56-1.62 (m, 8H), 1.43-1.50 (m, 8H) ppm. ¹³C NMR, 145.61, 144.26, 143.36, 142.56, 138.18, 137.35, 130.18, 129.13, 129.06, 128.01, 127.17, 61.81, 38.03, 32.11, 31.56, 31.07, 26.23 ppm.

### Example 28

### Preparation of 5,5′,5″,5′″-(1,2,4,5-benzentetrayl)-tetrakis[pentanyl-(3-phenylpyridinium)]tetrabromide

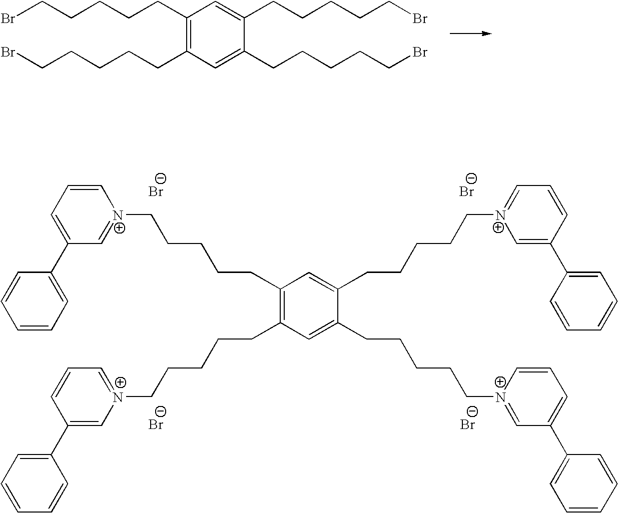

A mixture of 5,5′,5″,5′″-(1,2,4,5-benzentetrayl)tetrakis-[1-bromopentane] (330 mg, 0.49 mmol) and 3-phenyl-pyridine (356 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 488 mg of the title compound. Yield: 77%. ¹H NMR (300 MHz, CD₃OD) δ 9.48 (s, 4H), 9.05 (d, J=6.0 Hz, 4H), 8.83 (d, J=8.4 Hz, 4H), 8.15 (dd, 4H), 7.87-7.90 (m, 8H), 7.50-7.88 (m, 12H), 6.85 (s, 2H), 4.80 (t, J=7.5 Hz, 8H), 2.48-2.54 (m, 8H), 2.08-2.14 (m, 8H), 1.45-1.62 (m, 16H) ppm. ¹³C NMR, 142.92, 142.78, 142.56, 141.18, 137.27, 133.26, 130.34, 130.13, 129.65, 128.36, 127.61, 62.05, 32.03, 31.70, 30.98, 26.20 ppm.

### Example 29

### Preparation of 5,5′,5″,5′″-(1,2,4,5-benzentetrayl)-tetrakis[pentanyl-(5,6,7,8-tetrahydroisoquinolinium)]tetrabromide

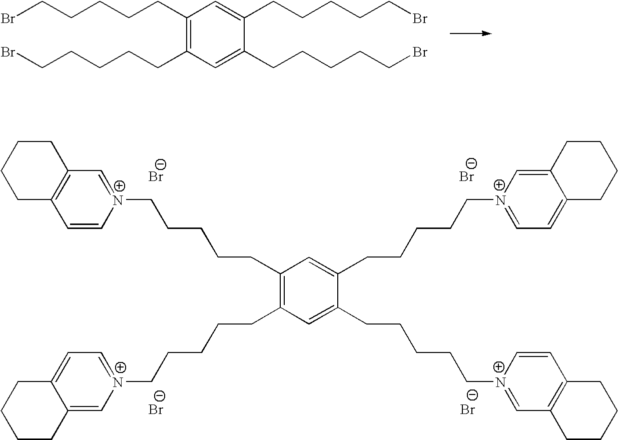

A mixture of 5,5′,5″,5′″-(1,2,4,5-benzentetrayl)-tetrakis-[1-bromopentane] (330 mg, 0.49 mmol) and 5,6,7,8-tetrahydroisoqinoline (306 mg, 2.3 mmol) was heated at 60-70° C. for 18 hrs. The resulted mixture was treated with diethyl ether and then dissolved in water (15 mL), the aqueous solution was extracted extensively with chloroform (30 mL×5). Water was removed through lyophilization to afford 455 mg of the title compound. Yield: 77%. ¹H NMR (300 MHz, CD₃OD) δ 8.94 (s, 4H), 8.78 (d, J=6.3 Hz, 4H), 7.83 (d, J=6.3 Hz, 4H), 6.94 (s, 2H), 4.65 (t, J=7.5 Hz, 8H), 3.07 (br, 8H), 3.00 (br, 8H), 2.58 (m, 8H), 2.10 (m, 8H), 1.90 (br, 16H), 1.63 (br, 8H), 1.50 (br, 8H) ppm. ¹³C NMR, 158.33, 143.92, 140.37, 138.79, 137.41, 130.16, 128.02, 60.90, 32.17, 31.52, 31.21, 29.54, 26.41, 26.32, 21.37 ppm.

What is claimed is:

1. A method of treating cancer or AIDS in a subject in need thereof comprising administering a pharmaceutically acceptable amount of a compound of Formula (I) 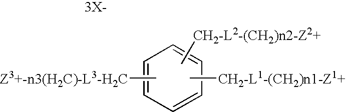
wherein the three side chains attached to the phenyl ring are connected to the 1, 2, and 3 positions; the 1, 2, and 4 positions; or the 1, 3, and 5 positions of the phenyl ring;
wherein:
each X⁻ is independently an organic or inorganic anion;
n1, n2, and n3 are each independently 1, 2, or 3
L¹, L², and L³ are each independently selected from the group consisting of —CH₂—CH₂—, cis —CH═CH—, trans —CH═CH—, and —C≡C—;
Z¹, Z², and Z³ are each independently a five or six membered heterocyclic or heteroaryl ring attached through N+ as shown below 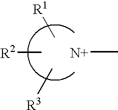
wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

2. The method of claim 1, wherein n1, n2, and n3 are 2.

3. The method of claim 1, wherein L¹, L², and L³ are each independently —CH₂CH₂— or —C≡C—.

4. The method of claim 3, wherein L¹, L², and L³ are the same.

5. The method of claim 1, wherein Z¹, Z², and Z³ are each independently pyridinyl rings attached through N+ as shown below 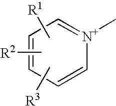
wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

6. The method of claim 5, wherein two of R¹, R², and R³ are hydrogen and one is alkyl, aryl, alkylaryl, arylalkyl, heterocyclic, alkylheterocyclic, heterocyclicalkyl, alkylheteroaryl, or heteroarylalkyl

7. The method of claim 5, wherein one of R¹, R², and R³ is hydrogen and two of R¹, R², and R³ together with the atoms to which they are attached form a six membered aryl ring.

8. The method of claim 5, wherein Z¹, Z², and Z³ are the same.

9. The method of claim 5, wherein R¹, R², and R³ are each independently selected from hydrogen, alkyl, aryl, alkylaryl, arylalkyl, or two of R¹, R², and R³ together with the carbon atoms to which they are attached form a six membered aryl ring.

10. The method of claim 1, wherein the compound of formula (I) is 1,3,5-tri-[5-(1-quinolinum)-pent-1-yn-1-yl]-benzene tribromide.

11. A method of treating cancer or AIDS in a subject in need thereof comprising administering a pharmaceutically acceptable amount of a compound of Formula (II) 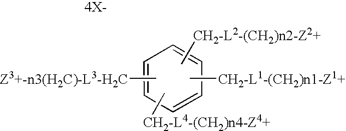
wherein the four side chains attached to the phenyl ring are connected to the 1, 2, 3, and 4 positions; the 1, 3, 4, and 5 positions; or the 1, 2, 4, and 5 positions of the phenyl ring;
wherein:
each X⁻ is independently an organic or inorganic anion;
n1, n2, n3 and n4 are each independently 1, 2, or 3
L¹, L², L³ and L⁴ are each independently selected from the group consisting of —CH₂CH₂—, cis —CH═CH—, trans —CH═CH—, and —C≡C—;
Z¹, Z², Z³ and Z⁴ are each independently a five or six membered heterocyclic or heteroaryl ring attached through N+ as shown below 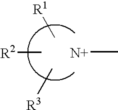
wherein R¹, R², and R³ are each independently selected from hydrogen, alkyl, cycloalkyl, alkylcycloalkyl, cycloalkylalkyl, alkenyl, alkynyl, aryl, alkylaryl, arylalkyl, arylalkenyl, arylalkynyl, heterocyclic, alkylheterocyclic, heterocyclicalkyl, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

12. The method of claim 11, wherein n1, n2, n3 and n4 are 2.

13. The method of claim 11, wherein L¹, L², L³ and L⁴ are each independently —CH₂CH₂— or —C≡C—.

14. The method of claim 13, wherein L¹, L², L³ and L⁴ are the same.

15. The method of claim 11, wherein Z¹, Z², Z³ and Z⁴ are each independently pyridinyl rings attached through N+ as shown below: 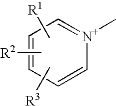
wherein R¹, R², and R³ are each independently selected from hydrogen, alkyl, cycloalkyl, alkenyl, alkynyl, aryl, alkylaryl, arylalkyl, arylalkenyl, arylalkynyl, heterocyclic, alkylheteroaryl, heteroarylalkyl, halo or two of R¹, R², and R³ together with the atoms to which they are attached form a three to six membered cycloalkyl, aryl, or heterocyclic with one to two hetero atoms in the ring.

16. The method of claim 15, wherein Z¹, Z², Z³ and Z⁴ are the same.

17. The method of claim 15, wherein two of R¹, R², and R³ are hydrogen and one is alkyl, aryl, alkylaryl, arylalkyl, heterocyclic, alkylheterocyclic, heterocyclicalkyl, alkylheteroaryl, or heteroarylalkyl.

18. The method of claim 15, wherein one of R¹, R², and R³ is hydrogen and two of R¹, R², and R³ together with the atoms to which they are attached form a six membered aryl.

19. The method of claim 15, wherein R¹, R², and R³ are each independently selected from hydrogen, alkyl, aryl, alkylaryl, arylalkyl, or two of R¹, R², and R³ together with the carbon atoms to which they are attached form a six membered aryl.

20. The method of claim 11, wherein the compound of formula (II) is 1,2,4,5-tetra-{5-[1-(3-benzyl)pyridinium]pent-1-yl}benzenetetrabromide.
